SEQUENCE LISTING (45 sequences)

SEQ ID NO: 1 - protein, 12 aa, Human immunodeficiency virus
GRKKRRQRRRPQ

SEQ ID NO: 2 - protein, 18 aa, Artificial Sequence
  Description of Artificial Sequence Synthetic peptide
KLALKLALKALKAALKLA

SEQ ID NO: 3 - protein, 15 aa, Drosophila sp.
RQIKWFQNRRMKWKK

SEQ ID NO: 4 - protein, 16 aa, Artificial Sequence
  Description of Artificial Sequence Synthetic peptide
RQIRIWFQNRRMRWRR

SEQ ID NO: 5 - protein, 17 aa, Artificial Sequence
  Description of Artificial Sequence Synthetic peptide
CSIPPEVKFNKPFVYLI

SEQ ID NO: 6 - protein, 13 aa, Rana temporaria
FVQWFSKFLGRIL

SEQ ID NO: 7 - protein, 10 aa, Maurus palmatus
GDCLPHLKLC

SEQ ID NO: 8 - protein, 18 aa, Mus sp.
LLIILRRRIRKQAHAHSK

SEQ ID NO: 9 - protein, 24 aa, Homo sapiens
LGTYTQDFNKFHTFPQTAIGVGAP

SEQ ID NO: 10 - protein, 30 aa, Unknown
  Description of Unknown Neurturin sequence
GAAEAAARVYDLGLRRLRQRRRLRRERVRA

SEQ ID NO: 11 - protein, 18 aa, Drosophila sp.
RQIKIWFQNRRMKWKKGG

SEQ ID NO: 12 - protein, 30 aa, Artificial Sequence
  Description of Artificial Sequence Synthetic polypeptide
RRRQRRKKRGGDIMGEWGNEIFGAIAGFLG

SEQ ID NO: 13 - protein, 11 aa, Human immunodeficiency virus
YGRKKRRQRRR

SEQ ID NO: 14 - protein, 18 aa, Unknown
  Description of Unknown SynB1 sequence
RGGRLSYSRRRFSTSTGR

SEQ ID NO: 15 - protein, 10 aa, Unknown
  Description of Unknown SynB3 sequence
RRLSYSRRRF

SEQ ID NO: 16 - protein, 11 aa, Artificial Sequence
  Description of Artificial Sequence Synthetic peptide
PIRRRKKLRRL

SEQ ID NO: 17 - protein, 12 aa, Artificial Sequence
  Description of Artificial Sequence Synthetic peptide
RRQRRTSKLMKR

SEQ ID NO: 18 - protein, 15 aa, Flock House virus
RRRRNRTRRNRRRVR

SEQ ID NO: 19 - protein, 19 aa, Brome Mosaic virus
KMTRAQRRAAARRNRWTAR

SEQ ID NO: 20 - protein, 13 aa, Human T-cell leukemia virus II
TRRQRTRRARRNR

SEQ ID NO: 21 - protein, 13 aa, Human immunodeficiency virus
GRKKRRQRRRPPQ

SEQ ID NO: 22 - protein, 13 aa, Artificial Sequence
  Description of Artificial Sequence Synthetic peptide
GRRRRRRRRRPPQ

SEQ ID NO: 23 - protein, 27 aa, Artificial Sequence
  Description of Artificial Sequence Synthetic peptide
GWTLNSAGYLLGKINLKALAALAKKIL

SEQ ID NO: 24 - protein, 27 aa, Homo sapiens
MGLGLHLLVLAAALQGAWSQPKKKRKV

SEQ ID NO: 25 - protein, 27 aa, Unknown
  Description of Unknown FBP sequence
GALFLGWLGAAGSTMGAWSQPKKKRKV

SEQ ID NO: 26 - protein, 27 aa, Artificial Sequence
  Description of Artificial Sequence Synthetic peptide
GALFLGFLGAAGSTMGAWSQPKKKRKV

SEQ ID NO: 27 - protein, 27 aa, Artificial Sequence
  Description of Artificial Sequence Synthetic peptide
GALFLGFLGAAGSTMGAWSQPKSKRKV

SEQ ID NO: 28 - protein, 21 aa, Artificial Sequence
  Description of Artificial Sequence Synthetic peptide
KETWWETWWTEWSQPKKKRKV

SEQ ID NO: 29 - protein, 21 aa, Artificial Sequence
  Description of Artificial Sequence Synthetic peptide
KETWFETWFTEWSQPKKKRKV

SEQ ID NO: 30 - protein, 17 aa, Artificial Sequence
  Description of Artificial Sequence Synthetic peptide
  MISC_FEATURE (1)..(17): This sequence may encompass 4-17 residues
RRRRRRRRRRRRRRRRR

SEQ ID NO: 31 - protein, 17 aa, Artificial Sequence
  Description of Artificial Sequence Synthetic peptide
  MISC_FEATURE (1)..(17): This sequence may encompass 4-17 residues
KKKKKKKKKKKKKKKKK

SEQ ID NO: 32 - protein, 13 aa, Artificial Sequence
  Description of Artificial Sequence Synthetic peptide
  MOD_RES (2)..(2): Aminocaproic acid
  MOD_RES (4)..(4): Aminocaproic acid
  MOD_RES (6)..(6): Aminocaproic acid
  MOD_RES (8)..(8): Aminocaproic acid
  MOD_RES (10)..(10): Aminocaproic acid
  MOD_RES (12)..(12): Aminocaproic acid
RXRXRXRXRXRXR

SEQ ID NO: 33 - protein, 13 aa, Artificial Sequence
  Description of Artificial Sequence Synthetic peptide
  MOD_RES (2)..(2): Aminobutyric acid
  MOD_RES (4)..(4): Aminobutyric acid
  MOD_RES (6)..(6): Aminobutyric acid
  MOD_RES (8)..(8): Aminobutyric acid
  MOD_RES (10)..(10): Aminobutyric acid
  MOD_RES (12)..(12): Aminobutyric acid
RXRXRXRXRXRXR

SEQ ID NO: 34 - protein, 13 aa, Artificial Sequence
  Description of Artificial Sequence Synthetic peptide
RGRGRGRGRGRGR

SEQ ID NO: 35 - protein, 13 aa, Artificial Sequence
  Description of Artificial Sequence Synthetic peptide
RMRMRMRMRMRMR

SEQ ID NO: 36 - protein, 13 aa, Artificial Sequence
  Description of Artificial Sequence Synthetic peptide
RTRTRTRTRTRTR

SEQ ID NO: 37 - protein, 13 aa, Artificial Sequence
  Description of Artificial Sequence Synthetic peptide
RSRSRSRSRSRSR

SEQ ID NO: 38 - protein, 10 aa, Artificial Sequence
  Description of Artificial Sequence Synthetic peptide
RRRRRRRRRR

SEQ ID NO: 39 - protein, 13 aa, Artificial Sequence
  Description of Artificial Sequence Synthetic peptide
RARARARARARAR

SEQ ID NO: 40 - protein, 7 aa, Artificial Sequence
  Description of Artificial Sequence Synthetic peptide
RRRRRRR

SEQ ID NO: 41 - DNA, 18 bp, Artificial Sequence
  Description of Artificial Sequence Synthetic oligonucleotide
gcacgggcagcttgccgg

SEQ ID NO: 42 - DNA, 20 bp, Artificial Sequence
  Description of Artificial Sequence Synthetic oligonucleotide
gctgaagcactgcacgccat

SEQ ID NO: 43 - DNA, 100 bp, Artificial Sequence
  Description of Artificial Sequence Synthetic polynucleotide
tcatgtggtcggggtagcggctgaagcactgcacgccatgggtcagggtggtcacgagggtgggccagggcaccggcagcttgccggtggtgcagatgaa

SEQ ID NO: 44 - DNA, 20 bp, Artificial Sequence
  Description of Artificial Sequence Synthetic oligonucleotide
aagtaaaacctctacaaatg

SEQ ID NO: 45 - DNA, 20 bp, Artificial Sequence
  Description of Artificial Sequence Synthetic oligonucleotide
cgttaatcgcgtataatacg

(19) United States
(12) Patent Application Publication
(10) Pub. No.: US 2022/0177494 A1
(43) Pub. Date: Jun. 9, 2022

(54) pH-RESPONSIVE SILICA METAL ORGANIC FRAMEWORK NANOPARTICLES FOR DELIVERY OF BIOACTIVE MOLECULES

(71) Applicant: Wisconsin Alumni Research Foundation, Madison, WI (US)

(72) Inventors: Shaoqin GONG, Middleton, WI (US); Yuyuan WANG, Madison, WI (US)

(73) Assignee: Wisconsin Alumni Research Foundation, Madison, WI (US)

(21) Appl. No.: 17/602,239

(22) PCT Filed: Apr. 8, 2020
(86) PCT No.: PCT/US2020/027284

§ 371 Date: Oct. 7, 2021

Related U.S. Application Data
(60) Provisional application No. 62/830,612, filed on Apr. 8, 2019.

(51) Int. Cl. C07F 3/06; A61K 47/69; A61K 31/704; A61K 9/51; A61K 9/00

(52) U.S. Cl. CPC C07F 3/06; A61K 47/6929; A61K 31/704; B82Y 5/00; A61K 9/5192; A61K 9/0019; A61K 9/0048; A61K 9/5146

(57) ABSTRACT

Provided herein are silica metal organic framework (SMOF) nanoparticles that are pH-responsive for delivery of bioactive molecules. The nanoparticles include a organosilica network comprising a plurality of imidazolyl and/or carboxyl groups; a metal organic framework component comprising a transition metal coordinated to a coordinating ligand, wherein the transition metal is selected from the group consisting of zinc, iron, zirconium, copper, and cobalt, and the coordinating ligand is selected from an imidazolate ligand or a carboxylate ligand; a bioactive payload selected from the group consisting of a hydrophilic drug, a polynucleic acid, a protein and a protein-polynucleic acid complex; and a surface-modifying polymer conjugated to the same or a different organosilica network and forming at least part of an exterior surface of the nanoparticle, wherein the surface-modifying polymer is selected from polyethylene glycol and/or a polyzwitterion; and wherein the zinc also coordinates the imidazolyl or carboxyl group of the organosilica network.

### CROSS-REFERENCE TO RELATED PATENT APPLICATIONS

This application is a U.S. National Phase Application under 35 U.S.C. § 371 of International Application No. PCT/US2020/027284, filed Apr. 8, 2020, which claims the benefit of priority to U.S. Provisional Patent Application No. 62/830,612, filed Apr. 8, 2019, the entire disclosure of each which are hereby incorporated by reference in their entireties for any and all purposes.

### SEQUENCE LISTING

The instant application contains a Sequence Listing which has been submitted electronically in ASCII format and is hereby incorporated by reference in its entirety. Said ASCII copy, created on May 7, 2020, is named 032026-1428_SL.txt and is 13,216 bytes in size.

### FIELD

The present technology relates generally to the field of nanoplatform delivery systems. The delivery systems include a silica metal organic framework nanoparticle carrying a payload of bioactive molecules. The nanoparticles are pH-responsive, allowing them to efficiently deliver hydrophilic drugs, polynucleic acids and complexes of proteins and nucleic acids to cells. Methods of preparing and using the nanoparticles are also provided.

### BACKGROUND

Notwithstanding myriad advances in drug delivery over the years, many types of bioactive compounds are difficult to safely deliver at concentrations high enough to provide the desired therapeutic and other effects. Bioactive compounds that remain challenging to deliver to the patient range from hydrophilic small molecule therapeutics such as doxorubicin (or the salt thereof) (an anticancer agent) to large biomolecules such as polynucleic acids, proteins and complexes of both proposed for gene therapy. In particular, clinical use of gene therapy has been limited due to various technical barriers, particularly, the lack of safe and efficient gene delivery systems.

Both plasmid DNA (DNA) and messenger RNA (mRNA) have been widely investigated for gene therapy. Both DNA and mRNA can be used to express functional proteins. To function, mRNA needs to reach the cytosol of the target cell, while DNA usually translocates to the cell nucleus for transgene expression. Both DNA and mRNA can result in relatively safe and rapid protein production for disease treatment. However, due to their relatively large sizes and high negative charge densities, naked DNA and mRNA exhibit low cellular uptake efficiency. Furthermore, naked DNA and mRNA are also susceptible to chemical degradation.

The clustered regularly interspaced short palindromic repeats (CRISPR)-Cas9 systems are powerful tools for genome editing. The Cas9/sgRNA ribonucleoprotein (RNP) can knock-out a target gene with high efficiency and specificity. Moreover, the combination of RNP and a DNA repair template (e.g., single-stranded donor oligonucleotide (ssODN) or donor polynucleic acid up to 2 kb) can achieve precise genome editing to incorporate sequences from the ssODN. However, safe and efficient delivery of RNP and RNP+donor DNA remains as a significant challenge for their potential application owing to their relatively large and complex structures. Similar to DNA and mRNA, unpackaged RNP and RNP+donor DNA are also susceptible to chemical degradation. Furthermore, in comparison to DNA and mRNA delivery, the delivery of protein/nucleic acid complexes such as RNP and RNP+donor DNA is even more challenging due to the mixed charges (e.g., positively charged Cas9 protein and negatively charged sgRNA and ssODN) and more sophisticated structures.

### SUMMARY OF THE INVENTION

The present technology provides a new nanoplatform for delivering bioactive payloads to cells. The nanoplatform comprises silica metal organic framework hybrid nanoparticles. Thus in one aspect, the present technology provides nanoparticles comprising: an organosilica network (e.g., polysiloxane) including a plurality of imidazolyl groups and/or carboxyl groups. The organosilica network further includes a plurality of surface-modifying moieties selected from the group consisting of polyethylene glycol (PEG), a polycation, a polyzwitterion, or functional groups that form cations at a pH of 8 or below. The nanoparticles also include metal organic framework components that include a transition metal ion coordinated to a coordinating ligand, wherein the transition metal ion is selected from the group consisting of zinc, iron, zirconium, copper, and cobalt ions, and the coordinating ligand is selected from an imidazolate ligand or a carboxylate ligand. The nanoparticles further include a bioactive payload selected from the group consisting of a hydrophilic drug, a polynucleic acid, a protein and a protein-polynucleic acid complex. The nanoparticle has an exterior surface with a plurality of surface-modifying groups. Nanoparticles of the present technology provide for comparable or higher loading and comparable or more efficient delivery of the bioactive payload along with lower toxicity than some traditional delivery platforms such as Lipofectamine. Further, with the present platform, there is no need to conjugate the polynucleic acid to the protein as in, e.g., S1mplex. The present technology also provides methods of making and using the new nanoparticles.

### BRIEF DESCRIPTION OF THE DRAWINGS

FIGS. 1A-1C show schematic representations of an illustrative embodiment of the present technology. (FIG. 1A) Schematic for SMOF NP for the delivery of various hydrophilic payloads such as hydrophilic small molecular drugs, DNAs, mRNAs, proteins, and a combination thereof (e.g., Cas9/sgRNA (RNP) and RNP+ssODN). (FIG. 1B) Synthesis of SMOF NPs via a water-in-oil emulsion method. (FIG. 1C) Schematic illustration of the intracellular trafficking pathways of SMOF NPs. SMOF NP: silica-metal-organic-framework hybrid nanoparticle; 2-MIM: 2-methylimidazole; PEG: polyethylene glycol; TEOS: tetraethyl orthosilicate; TESPIC: N-(3-(triethoxysilyl)propyl)-1H-imidazole-2-carboxamide; APTES: (3-aminopropyl)triethoxysilane.

FIGS. 2A-2E. Characterization of SMOF NPs and intracellular trafficking. (FIG. 2A) TEM and (FIG. 2B) SEM micrographs of DNA-loaded SMOF NPs. (FIG. 2C) Size distribution of SMOF NPs measured by DLS. (FIG. 2D) Powder XRD spectra of SMOF NP, pure MOF NP, and pure silica NP synthesized via the water-in-oil emulsion method. (FIG. 2E) Intracellular trafficking of RNP-loaded SMOF NPs by CLSM. Colocalization of RNP and endo/lysosomes was studied at 0.5 h, 2 h, and 4 h post-treatment. Scale bar of (2E): 50 μm.

FIG. 3 shows energy-dispersive X-ray spectroscopy (EDS) spectrum for an illustrative embodiment of a SMOF NP of the present technology.

FIGS. 4A-4B. Delivery Efficiency of a hydrophilic drug by SMOF NPs and intracellular trafficking. (FIG. 4A) Flow cytometry results of HEK293 cells treated with free DOX.HCl, and DOX.HCl-loaded SMOF NPs (DOX.HCl concentration, 50 μg/ml) or medium alone (control) for 120 min. (FIG. 4B) Cytotoxicity of DOX.HCl-loaded SMOF NPs at different DOX.HCl concentrations (i.e., 6 and 12 μg/ml) after co-incubation with HEK293 cells for 48 h.

FIG. 5. Representative fluorescence microscopy images of HEK293 cells. Cells were treated with culture media (control), free DOX.HCl, and DOX.HCl-loaded SMOF NPs. Scale bar: 10 μm.

FIG. 6 shows the optimization of an illustrative embodiment of a DNA-loaded SMOF NP formulation using HEK 293 cells. (FIG. 6A) Optimization of the feed weight ratio of the payload over the SMOF reactants, feed weight ratio of the silica reactants to the MOF reactant, the emulsification method, and the effect of an additive (i.e., glycerol) in the aqueous phase. (FIG. 6B) Optimization of the molar ratio of the three silica reactants TEOS/APTES/TESPIC. The optimal SMOF NP formulation is highlighted by a black bar. NS: not significant; *: p<0.05; **: p<0.01; ***: p<0.001; ****: p<0.0001; n=3.

FIG. 7 Optimization of the mRNA-loaded SMOF NP formulations. The effects of (1) feed ratios of the payloads (i.e., mRNA) to the SMOF reactants by weight, (2) feed ratios of silica reactants over the MOF reactant by weight, (3) emulsification methods, and (4) glycerol in an aqueous phase were systematically investigated for mRNA-loaded SMOF NPs. The transfection efficiencies of the various formulations were evaluated by quantification of GFP-positive HEK 293 cells 48 h after treatments. NS: not significant; **: p<0.01; ***: p<0.001; ****: p<0.0001; n=3.

FIGS. 8A-8E. Delivery efficiency of nucleic acids and CRISPR-Cas9 genome editing machineries by SMOF NPs. Transfection efficiency of the (FIG. 8A) DNA- and (FIG. 8B) mRNA-loaded SMOF NPs in HEK293, HCT116, NHDF, and RAW264.7 cells. (FIG. 8C) Genome-editing efficiency of RNP-loaded SMOF NPs in GFP-expressing HEK 293 cells. (FIG. 8D) Precise gene correction efficiency of RNP+ssODN co-loaded SMOF NPs in BFP expressing HEK 293 cells. The precise gene correction efficiency of RNP+ssODN repair template converting the BFP to the GFP was assayed by flow cytometry for gain of GFP fluorescence. NS: not significant; *: p<0.05; **: p<0.01; ***: p<0.005; n=3. (FIG. 8E) Viability of HEK293 cells treated with Lip0 2000 and SMOF NPs with different concentrations. NS: not significant; ***: p<0.005; n=5.

FIGS. 9A-9D. SMOF NPs induced efficient genome editing in vivo in Ai14 mice via local administration. (FIG. 9A) The tdTomato locus in the Ai14 reporter mouse. A stop cassette containing 3 Ai14 sgRNA target sites prevents downstream tdTomato expression. RNP guided excision of the stop cassette results in tdTomato expression. (FIG. 9B) Illustration of SMOF NP subretinal injection targeting the RPE tissue. (FIG. 9C) Representative images of tdTomato+ signal (red) 13 days after subretinal SMOF-ATRA injection. The whole RPE layer was outlined with a white dotted line. Left: the left eye of Ai14 mouse injected with negative control SMOF-ATRA (SMOF-ATRA encapsulating RNP with negative control sgRNA). Middle: right eye of Ai14 mouse injected with SMOF-ATRA encapsulating RNP targeting the Ai14 stop cassette. Right: zoom-in image of genome-edited RPE tissue induced by RNP-loaded SMOF-ATRA. (FIG. 9D) Genome editing efficiency as quantified by percent of the area of whole RPE tissue with tdTomato+ signals. n=4 for all conditions. *: p<0.05.

### DETAILED DESCRIPTION

The following terms are used throughout as defined below. All other terms and phrases used herein have their ordinary meanings as one of skill in the art would understand.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having (unless indicated otherwise) from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The term “carboxyl” or “carboxylate” as used herein refers to a —COOH group or the salt thereof.

The term “imidazolyl” or “imidazolate” as used herein refers to a heterocyclic organic compound containing two nitrogen atoms separated by a carbon atom in a five-membered ring, (i.e., 1,3-diazole) or the salt thereof. Representative substituted imidazolyl groups may be substituted one or more times with substituents such as those listed above.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “hydroxyl” as used herein can refer to —OH or its ionized form, —O⁻. A “hydroxyalkyl” group is a hydroxyl-substituted alkyl group, such as HO—CH₂—.

As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 3rd Ed. (John Wiley & Sons, Inc., New York). Amino protecting groups include, but are not limited to, mesitylenesulfonyl (Mts), benzyloxycarbonyl (Cbz or Z), t-butyloxycarbonyl (Boc), t-butyldimethylsilyl (TBS or TBDMS), 9-fluorenylmethyloxycarbonyl (Fmoc), allyloxycarbonyl (Alloc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, α,α-dimethyldimethoxybenzyloxycarbonyl (DDZ), 5-bromo-7-nitroindolinyl, and the like. Amino protecting groups susceptible to acid-mediated removal include but are not limited to Boc and TBDMS. Amino protecting groups resistant to acid-mediated removal and susceptible to hydrogen-mediated removal include but are not limited to Alloc, Cbz, nitro, and 2-chlorobenzyloxycarbonyl.

As used herein, “Cas9 polypeptide” (also known as “Cas9”) refers to Cas9 proteins and variants thereof having nuclease activity, as well as fusion proteins containing such Cas9 proteins and variants thereof. The fused proteins may include those that modify the epigenome or control transcriptional activity. The variants may include deletions or additions, such as, e.g., addition of one, two, or more nuclear localization sequences (such as from SV40 and others known in the art), e.g., 1, 2, 3, 4, 5, 6, 7, 8, 9, 10 such sequences or a range between and including any two of the foregoing values. In some embodiments the Cas9 polypeptide is a Cas9 protein found in a type II CRISPR-associated system. Suitable Cas9 polypeptides that may be used in the present technology include, but are not limited to Cas9 protein from Streptococcus pyogenes (Sp. Cas9), F. novicida, S. aureus, S. thermophiles, N. meningitidis, and variants thereof. In some embodiments, the Cas9 polypeptide is a wild-type Cas9, a nickase, or comprises a nuclease inactivated (dCas9) protein. In some embodiments, the Cas9 polypeptide is a fusion protein comprising dCas9. In some embodiments, the fusion protein comprises a transcriptional activator (e.g., VP64), a transcriptional repressor (e.g., KRAB, SID) a nuclease domain (e.g., FokI), base editor (e.g., adenine base editors, ABE), a recombinase domain (e.g., Hin, Gin, or Tn3), a deaminase (e.g., a cytidine deaminase or an adenosine deaminase) or an epigenetic modifier domain (e.g., TET1, p300). In some embodiments, the Cas9 polypeptide includes variants with at least 85% sequence identity, at least 90% sequence identity, at least 95% sequence identity, or even 96%, 97%, 98%, or 99% sequence identity to the wild type Cas9. Accordingly, a wide variety of Cas9 polypeptides may be used as formation of the nanoparticle is not sequence dependent so long as the Cas9 polypeptide can complex with nucleic acids and the resulting RNP may associate with the other constituents of the present nanoparticles. Other suitable Cas9 polypeptides may be found in Karvelis, G. et al. “Harnessing the natural diversity and in vitro evolution of Cas9 to expand the genome editing toolbox,” Current Opinion in Microbiology 37: 88-94 (2017); Komor, A. C. et al. “CRISPR-Based Technologies for the Manipulation of Eukaryotic Genomes,” Cell 168:20-36 (2017); and Murovec, J. et al. “New variants of CRISPR RNA-guided genome editing enzymes,” Plant Biotechnol. J. 15:917-26 (2017), each of which is incorporated by reference herein in their entirety.

“Hydrophilic drug” as used herein refers to non-polymeric molecules that exert a therapeutic effect in an animal in the treatment of a disorder, disease or condition, and are soluble in water at 25° C. to at least 1 mg/mL. In any embodiments, a hydrophilic drug may have water solubility of at least 5 mg/ml, at least 10 mg/mL, at least 15 mg/mL, at least 20 mg/mL or at least 33 mg/mL at 25° C. Hence, hydrophilic drugs include doxorubicin hydrochloride (“DOX.HCl”), Y-27632 dihydrochloride ((1R,4r)-4-((R)-1-aminoethyl)-N-(pyridin-4-yl)cyclohexane-1-carboxamide dihydrochloride; a Rho-Associated Coil Kinase (ROCK) inhibitor). Hydrophilic drugs do not include biomacromolecule therapeutics such as DNA, mRNA, proteins or complexes of such biomolecules (e.g., cas9/sgRNA).

“Metal organic framework” or “MOF” as used herein refers to the three-dimensional, porous, crystalline structure formed by metal ions and small organic ligands that coordinate to the metal ions. Thus, a “metal organic framework component” refers collectively to the individual component parts of the MOF, i.e., a metal ion and a coordinating ligand. For example, a zinc ion and 2-methylimidazole would be, respectively, the metal ion and coordinating ligand of the metal organic framework component for the MOF, zeolitic imidazolate framework-8, i.e., ZIF-8.

“Molecular weight” as used herein with respect to polymers refers to number-average molecular weights (Mₙ) and can be determined by techniques well known in the art including gel permeation chromatography (GPC). GPC analysis can be performed, for example, on a D6000M column calibrated with poly(methyl methacrylate) (PMMA) using triple detectors including a refractive index (RI) detector, a viscometer detector, and a light scattering detector, and N,N′-dimethylformamide (DMF) as the eluent. “Molecular weight” in reference to small molecules and not polymers is actual molecular weight, not number-average molecular weight.

“Organosilica network” refers to polysiloxane polymers that are at least partly cross-linked to each other. Polysiloxanes of the present technology comprise repeating silicon-containing substructures of which a fraction (e.g., about 0.01 mol % to about 40 mol % including about 0.1 mol % to about 35 mol %, about 1.0 mol % to about 25 mol %, or about 15 mol % to about 25 mol %) of the repeating silicon-containing substructures include one or more Si—R bonds. Herein each R is independently a C₁₋₁₀ group such as, e.g., alkyl, aryl (e.g., phenyl), aralkyl (e.g., benzyl, phenethyl) groups optionally substituted with groups as defined herein including amino, carbonyl, ester, amides, and/or imidazoles). The position of the organic R groups can be terminal (Si—R) meaning that the R group is monovalently bound to a silicon atom, or bridging (Si—R′—Si) meaning R′ is the divalent form of the R group bound to two separate silicon atoms, e.g. as part of the polysiloxane backbone or as a cross-link between polysiloxane polymers. The organosilica network may include silicon atoms with two and/or three and/or four polymeric attachment points (i.e., to other siloxy, Si—R, or -bridging groups, e.g., —R′—Si). In some embodiments, the organosilica network may be formed by condensing materials from silicon alkoxide precursors with terminating organic groups, as well as precursors with organic bridging groups between Si centers. In some embodiments, the organosilica network may comprise silicon-containing substructures having the structure —O—Si(R^{a})(R^{b})—, wherein one or both of R^{a} and R^{b} are independently the R group defined above or more preferably a C₁₋₆ alkyl group optionally substituted with groups as defined herein (e.g, amino, carboxylate, amides, imidazoles). One of R^{a} and R^{b} may be a C₁₋₆ alkoxy group.

The phrase “targeting ligand” refers to a ligand that binds to “a targeted receptor” that distinguishes the cell being targeted from other cells. The ligands may be capable of binding due to expression or preferential expression of a receptor for the ligand, accessible for ligand binding, on the target cells. Examples of such ligands include GE11 peptide, anti-EGFR nanobody, cRGD ((cyclo (RGDfC)), KE108 peptide, octreotide, glucose, folic acid, prostate-specific membrane antigen (PSMA) aptamer, TRC105, a human/murine chimeric IgG1 monoclonal antibody, mannose, and cholera toxin B (CTB). Additional examples of such ligands include Rituximab, Trastuzumab, Bevacizumab, Alemtuzumab, Panitumumab, RGD, DARPins, RNA aptamers, DNA aptamers, analogs of folic acid and other folate receptor-binding molecules, lectins, other vitamins, peptide ligands identified from library screens, tumor-specific peptides, tumor-specific aptamers, tumor-specific carbohydrates, tumor-specific monoclonal or polyclonal antibodies, Fab or scFv (i.e., a single chain variable region) fragments of antibodies such as, for example, an Fab fragment of an antibody directed to EphA2 or other proteins specifically expressed or uniquely accessible on metastatic cancer cells, small organic molecules derived from combinatorial libraries, growth factors, such as EGF, FGF, insulin, and insulin-like growth factors, and homologous polypeptides, somatostatin and its analogs, transferrin, lipoprotein complexes, bile salts, selecting, steroid hormones, Arg-Gly-Asp containing peptides, microtubule-associated sequence (MTAS), various galectins, δ-opioid receptor ligands, cholecystokinin A receptor ligands, ligands specific for angiotensin AT1 or AT2 receptors, peroxisome proliferator-activated receptor γ ligands, β-lactam antibiotics, small organic molecules including antimicrobial drugs, and other molecules that bind specifically to a receptor preferentially expressed on the surface of targeted cells or on an infectious organism, or fragments of any of these molecules.

The phrase “a targeted receptor” refers to a receptor expressed by a cell that is capable of binding a cell targeting ligand. The receptor may be expressed on the surface of the cell. The receptor may be a transmembrane receptor. Examples of such targeted receptors include EGFR, αᵥβ₃ integrin, somatostatin receptor, folate receptor, prostate-specific membrane antigen, CD105, mannose receptor, estrogen receptor, and GM1 ganglioside.

In some embodiments, cell penetrating peptides may also be attached to one or more PEG terminal groups in place of or in addition to the targeting ligand. A “cell penetrating peptide” (CPP), also referred to as a “protein transduction domain” (PTD), a “membrane translocating sequence,” and a “Trojan peptide”, refers to a short peptide (e.g., from 4 to about 40 amino acids) that has the ability to translocate across a cellular membrane to gain access to the interior of a cell and to carry into the cells a variety of covalently and noncovalently conjugated cargoes, including proteins, oligonucleotides, and liposomes. They are typically highly cationic and rich in arginine and lysine amino acids. Examples of such peptides include TAT cell penetrating peptide (GRKKRRQRRRPQ (SEQ ID NO: 1)); MAP (KLALKLALKALKAALKLA (SEQ ID NO: 2)); Penetratin or Antenapedia PTD (RQIKWFQNRRMKWKK (SEQ ID NO: 3)); Penetratin-Arg: (RQIRIWFQNRRMRWRR (SEQ ID NO: 4)); antitrypsin (358-374): (CSIPPEVKFNKPFVYLI (SEQ ID NO: 5)); Temporin L: (FVQWFSKFLGRIL-NH2 (SEQ ID NO: 6)); Maurocalcine: (GDC(acm) LPHLKLC (SEQ ID NO: 7)); pVEC (Cadherin-5): (LLIILRRRIRKQAHAHSK (SEQ ID NO: 8)); Calcitonin: (LGTYTQDFNKFHTFPQTAIGVGAP (SEQ ID NO: 9)); Neurturin: (GAAEAAARVYDLGLRRLRQRRRLRRERVRA (SEQ ID NO: 10)); Penetratin: (RQIKIWFQNRRMKWKKGG (SEQ ID NO: 11)); TAT-HA2 Fusion Peptide: (RRRQRRKKRGGDIMGEWGNEIFGAIAGFLG (SEQ ID NO: 12)); TAT (47-57) (YGRKKRRQRRR (SEQ ID NO: 13)); SynB1 (RGGRLSYSRRRFSTSTGR (SEQ ID NO: 14)); SynB3 (RRLSYSRRRF (SEQ ID NO: 15)); PTD-4 (PIRRRKKLRRL (SEQ ID NO: 16)); PTD-5 (RRQRRTSKLMKR (SEQ ID NO: 17)); FHV Coat-(35-49) (RRRRNRTRRNRRRVR (SEQ ID NO: 18)); BMV Gag-(7-25) (KMTRAQRRAAARRNRWTAR (SEQ ID NO: 19)); HTLV-II Rex-(4-16) (TRRQRTRRARRNR (SEQ ID NO: 20)); HIV-1 Tat (48-60) or D-Tat (GRKKRRQRRRPPQ (SEQ ID NO: 21)); R9-Tat (GRRRRRRRRRPPQ (SEQ ID NO: 22)); Transportan (GWTLNSAGYLLGKINLKALAALAKKIL chimera (SEQ ID NO: 23)); SBP or Human P1 (MGLGLHLLVLAAALQGAWSQPKKKRKV (SEQ ID NO: 24)); FBP (GALFLGWLGAAGSTMGAWSQPKKKRKV (SEQ ID NO: 25)); MPG (ac-GALFLGFLGAAGSTMGAWSQPKKKRKV-cya (SEQ ID NO: 26) (wherein cya is cysteamine)); MPG(ΔNLS) (ac-GALFLGFLGAAGSTMGAWSQPKSKRKV-cya (SEQ ID NO: 27)); Pep-1 or Pep-1-Cysteamine (ac-KETWWETWWTEWSQPKKKRKV-cya (SEQ ID NO: 28)); Pep-2 (ac-KETWFETWFTEWSQPKKKRKV-cya (SEQ ID NO: 29)); Periodic sequences, Polyarginines (R×N (4<N<17) chimera (SEQ ID NO: 30)); Polylysines (K×N (4<N<17) chimera (SEQ ID NO: 31)); (RAca)6R (SEQ ID NO: 32); (RAbu)6R (SEQ ID NO: 33); (RG)6R (SEQ ID NO: 34); (RM)6R (SEQ ID NO: 35); (RT)6R (SEQ ID NO: 36); (RS)6R (SEQ ID NO: 37); R10 (SEQ ID NO: 38); (RA)6R (SEQ ID NO: 39); and R7 (SEQ ID NO: 40).

A “dye” refers to small organic molecules having a molecular weight (actual, not number average) of 2,000 Da or less or a protein which is able to emit light. Non-limiting examples of dyes include fluorophores, chemiluminescent or phosphorescent entities. For example, dyes useful in the present technology include but are not limited to cyanine dyes (e.g., Cy2, Cy3, Cy5, Cy5.5, Cy7, and sulfonated versions thereof), fluorescein isothiocyanate (FITC), ALEXA FLUOR® dyes (e.g., ALEXA FLUOR® 488, 546, or 633), DYLIGHT® dyes (e.g., DYLIGHT® 350, 405, 488, 550, 594, 633, 650, 680, 755, or 800) or fluorescent proteins such as GFP (Green Fluorescent Protein).

The present technology provides hybrid silica metal organic framework nanoparticles that include a bioactive payload that can be safely delivered to the cells. The nanoparticles include a organosilica network comprising a plurality of imidazolyl and/or carboxyl groups. The organosilica network further comprises a plurality of surface-modifying moieties selected from the group consisting of polyethylene glycol (PEG), a polycation, a polyzwitterion, or functional groups that form cations at a pH of 8 or below. The nanoparticle is also made up of metal organic framework components having a transition metal ion coordinated to a coordinating ligand, wherein the transition metal is selected from the group consisting of zinc, iron, zirconium, copper, and cobalt, and the coordinating ligand is selected from an imidazolate ligand or a carboxylate ligand. The present nanoparticles also include a bioactive payload selected from the group consisting of a hydrophilic drug, a polynucleic acid, a protein and a protein-polynucleic acid complex. The nanoparticle includes an exterior surface with a plurality of surface-modifying groups as described herein.

Nanoparticles of the present technology employ a organosilica network (the silica) to allow far greater functionalization of the nanoparticle than can be readily achieved with metal organic frameworks alone. For example, the organosilica network of the present nanoparticles include imidazolyl or carboxyl groups that, like the organic framework component of a MOF can coordinate to the metals of the present nanoparticles. It will be appreciated that when the transition metal ion is actually coordinated to the imidazolyl or carboxyl groups of the organosilica network, the foregoing groups may be but are not necessarily in their anionic imidazolate or carboxylate forms. However, to distinguish these groups from the MOF coordinating ligands that are also part of the nanoparticles, they shall be referred to as imidazolyl and carboxyl groups, and such designation shall encompass their neutral or charged forms. In any embodiments, the organosilica network may include imidazolyl groups.

The organosilica network may further include a plurality of surface-modifying moieties. While not wishing to be bound by theory, the surface-modifying moieties may serve to solubilize and/or stabilize the nanoparticles. The surface-modifying moieties may be selected from the group consisting of polyethylene glycol (PEG), a polycation, a polyzwitterion, or functional groups that form cations at a pH of 8 or below. In any embodiments, the surface-modifying moieties may include functional groups that form cations at a pH of 8 or below such as, but not limited to, amino, guanidine, and pyridyl groups. In any embodiments, the surface-modifying groups may include amino groups. These amino groups, when protonated, provide positive charge on the nanoparticles' exterior surfaces, and can also be used to attach other surface-modifying moieties to the nanoparticles such as PEG, polycations and polyzwitterions. The polycations used herein are polymers bearing protonateable organic functional groups such as amines, imines, amidines, guanidines, and the like. Polycations suitable for use in the present technology include polyethyleneimine (PEI), polylysine, and polyamidoamine (PAMAM). The surface-modifying moieties used in the present nanoparticles may also include polyethylene glycol (PEG) or polyzwitterions, which are likewise polymers. In any embodiments, the polyzwitterions may be poly(carboxybetaine methacrylate), poly(sulfobetain methacrylate), and/or poly(2-methacryloyloxyethyl phosphorylcholine).

In any embodiments, the surface-modifying moieties that are polymeric (e.g., PEG, polycation, and polyzwitterion) may have a Mn of about 1,000 to about 50,000 Da. For example, the PEG, polycation, or polyzwitterion may have a Mn of about 1,000, about 2,000, about 3,000, about, 4,000, about 5,000, about 7,500, about 10,000, about 15,000, about 20,000, about 30,000, about 40,000, about 50,000 Da or a value within a range between and including any two of the foregoing values. For example, the PEG, polycation, or polyzwitterion may have a Mn of about 2,000 to about 10,000 Da.

The present nanoparticles may also include a targeting ligand and/or an imaging agent attached to the organosilica network. The targeting ligand and/or imaging agent may be attached to the organosilica network via bonds to amino groups in the organosilica network. By way of a non-limiting example, the bonds may be amide bonds, N—C bonds, imino bonds and the like.

The present nanoparticles include a metal organic framework component as described above. Suitable metal ions that may be employed in the metal organic framework component include zinc, iron, zirconium, copper, and cobalt ions. In any embodiments the metal ion may be zinc ion or it may be iron ion. The coordinating ligand may be an imidazolate ligand or a carboxylate ligand as noted above. Imidazolate ligands are coordinating ligands that contain an imidazole group such as, e.g., imidazole itself, 2-methyl-imidazole, benzimidazole, or 5-methylbenzimidazole. Carboxylate ligands include, e.g., terephthalic acid, 2-methyl-pterphthalic acid, 2-hydroxy-terephthalic acid, and 2-amino-terephthalic acid. The imidazolate and carboxylate ions are typically but are not necessarily in their anionic forms. Those of skill in the art will recognize which ligands are suitable for use with a particular type of metal to form a metal organic framework component. By way of example only, zinc may be used with imidazolate ligands and iron may be used with carboxylate ligands, especially dicarboxylate ligands.

The weight ratio of organosilica network to metal organic framework component may vary. For example, it may range from 3:1 to 1:3. In any embodiments, the ratio may be for example, 3:1, 2:1, 3:2, 1:1, 2:3, 1:2, 1:3 or a value within a range between and including any two of the foregoing values. For example, the ratio may be 2:1 to 1:2. A person skilled in the art will be readily able to optimize the ratio of organosilica network to metal organic framework component for the delivery application at hand based on the present disclosure.

Suitable payloads for the present nanoparticle delivery systems include hydrophilic drugs, proteins, polynucleic acids and complexes of the two such as ribonucleoproteins (RNP), e.g., Cas9 with guide RNA. Examples of hydrophilic small molecule therapeutics include DOX.HCl and Y-27632 dihydrochloride. In any embodiments, the bioactive payload may include DOX.HCl, DNA, RNA (e.g., mRNA), ribonucleoprotein (RNP), and combinations of two or more thereof. In any embodiments, the bioactive payload may be selected from the group consisting of plasmid DNA (pDNA), single-stranded donor oligonucleotide (ssODN), complementary (cDNA), messenger RNA (mRNA), small interfering RNA (siRNA), microRNA (miRNA), short hairpin RNA (shRNA), single guide RNA (sgRNA), transfer RNA (tRNA), ribozymes, and combinations of two or more thereof. In certain embodiments, the bioactive payload may be selected from the group consisting of Cas9 RNP, RNP+ssODN where ssODN serves as a repair template, RNP+donor DNA up to 2 kb, and other Cas9-based protein/nucleic acid complexes. It will be appreciated that with the present nanoparticles, Cas9 or RNP need not be conjugated to any repair template as either may simply be mixed with the desired polynucleic acid instead during the nanoparticle formation process. NLS peptides may be used to direct payload to the nucleus if desired. For example, polynucleic acids as described herein as well as proteins such as Cas9 or RNP+ donor DNA complexes may be covalently tagged with NLS peptides using techniques well known in the art.

The present nanoparticles have a hydrodynamic diameter ranging from 10 nm to 500 nm. For example, they may have a hydrodynamic diameter of 10, 20, 30, 40, 50, 60, 70, 80, 90, 100, 110, 130, 150, 170, 200, 250, 300, 350, 400, or 500 nm or a range between and including any two of the foregoing values. In any embodiments herein, they may have a hydrodynamic diameter of 20 to 200 nm or even 30 to 150 nm. In any embodiments, the hydrodynamic diameter may be an average hydrodynamic diameter or a median hydrodynamic diameter selected from the foregoing ranges.

In another aspect, the present technology provides methods of making the nanoparticles described herein. The methods include forming a nanoparticle comprising an organosilica network by adding organosilica network precursors and an organic framework component to an emulsion of water and an organic solvent, whereby the organosilica network precursors polymerize to form the organosilica network. The organosilica network precursors include imidazolyl groups and/or carboxyl groups and/or functional groups that form cations at a pH of 8 or below. The emulsion includes a metal ion and a bioactive payload, wherein the metal is selected from the group consisting of zinc, iron, zirconium, copper, and cobalt, and the bioactive payload selected from the group consisting of a hydrophilic drug, a polynucleic acid, a protein and a protein-polynucleic acid complex. The emulsion may be formed from any suitable organic solvents (including, e.g., alkanes, cycloalkanes, alcohols and non-ionic detergents and mixtures of any two or more thereof) and water. In any embodiment, the emulsion may include hexanol, cyclohexane, Triton X-100 (polyethylene glycol p-(1,1,3,3-tetramethylbutyl)-phenyl ether) and water. In any embodiments, the emulsion may be formed by any suitable methods such as shaking, vortexing, and sonication. The organosilica network precursors may include suitable orthosilicate monomers, siloxy imidazole monomers, and siloxy amine monomers or other siloxy monomers with functional groups that form cations at a pH of 8 or below. Thus, in any embodiments, the organosilica network precursors may include tetraethyl orthosilicate, N-(3-(triethoxysilyl)propyl)-1H-imidazole-2-carboxamide and (3-aminopropyl)triethoxysilane. The amounts of the organosilica precursors may range from 60-90 mol % orthosilicate monomers and/or 5-35 mol % each of siloxy imidazole monomers and siloxy amine monomers. In any embodiments, the present methods may further include attaching a surface-modifying moiety to the organosilica network, wherein the surface-modifying moiety is selected from the group consisting of PEG, a polycation, and a polyzwitterion. The surface-modifying moieties typically have a reactive group such as an electrophile or active ester or the like which can react with, e.g., a nucleophilic group on the organosilica network such as, but not limited to amino groups. The nanoparticles thus formed may be precipitated from solution with a suitable organic solvent, e.g., acetone.

In another aspect, the present technology provides methods of delivering a bioactive payload to a target cell for treatment such as nucleic acid delivery and genome editing machinery delivery for of a variety of eye diseases including diseases related to dysfunctioning retinal pigmented epithelial cells (RPE cells) in the eye (e.g., retinal degeneration and blindness) as well as other monogenic diseases of the eye, cancer treatment (e.g., chemotherapy, cancer immunotherapy), neuromuscular disease treatment (e.g., muscular dystrophy, peripheral neuropathy), neurological disease treatment (e.g., Parkinson's disease, Alzheimer's disease), and vaccines. The methods include exposing the targeted cell to any of the herein-described nanoparticles. The methods include both in vitro and in vivo methods. For example, the methods may include administering any of the herein-described nanoparticles to a subject in need thereof (i.e., a subject in need of the bioactive payload to be delivered by the nanoparticles). As used herein, a “subject” is a mammal, such as a cat, dog, rodent or primate. In some embodiments, the subject is a human. In some embodiments, the payload is any of those described herein, including but not limited to DOX.HCl, DNA, pDNA, mRNA, siRNA, Cas9 RNP, RNP+donor nucleic acids.

The compositions described herein can be formulated for various routes of administration, for example, by parenteral, intravitreal, intrathecal, intracerebroventricular, rectal, nasal, vaginal administration, direct injection into the target organ, or via implanted reservoir. Parenteral or systemic administration includes, but is not limited to, subcutaneous, intravenous, intraperitoneal, and intramuscular injections. The following dosage forms are given by way of example and should not be construed as limiting the instant present technology.

Injectable dosage forms generally include solutions or aqueous suspensions which may be prepared using a suitable dispersant or wetting agent and a suspending agent so long as such agents do not interfere with formation of the nanoparticles described herein. Injectable forms may be prepared with acceptable solvents or vehicles including, but not limited to sterilized water, phosphate buffer solution, Ringer's solution, 5% dextrose, or an isotonic aqueous saline solution.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference. Exemplary carriers and excipients may include but are not limited to USP sterile water, saline, buffers (e.g., phosphate, bicarbonate, etc.), tonicity agents (e.g., glycerol),

Specific dosages may be adjusted depending on conditions of disease, the age, body weight, general health conditions, sex, and diet of the subject, dose intervals, administration routes, excretion rate, and combinations of drug conjugates. Any of the above dosage forms containing effective amounts are well within the bounds of routine experimentation and therefore, well within the scope of the instant present technology. By way of example only, such dosages may be used to administer effective amounts of the present nanoparticles to the patient and may include 0.1, 0.2, 0.3, 0.4, 0.5, 0.75, 1.0, 1.5, 2.0, 2.5, 3.0, 4.0, 5.0, 6.0, 7.0, 8.0, 9.0, 10, 11, 12, 13, 14, 15 mg/kg or a range between and including any two of the forgoing values such as 0.1 to 15 mg/kg. Such amounts may be administered parenterally as described herein and may take place over a period of time including but not limited to 5 minutes, 10 minutes, 20 minutes, 30 minutes, 45 minutes, 1 hour, 2 hours, 3 hours, 5 hours, 10 hours, 12, hours, 15 hours, 20 hours, 24 hours or a range between and including any of the foregoing values. The frequency of administration may vary, for example, once per day, per 2 days, per 3 days, per week, per 10 days, per 2 weeks, or a range between and including any of the foregoing frequencies. Alternatively, the compositions may be administered once per day on 2, 3, 4, 5, 6 or 7 consecutive days. A complete regimen may thus be completed in only a few days or over the course of 1, 2, 3, 4 or more weeks.

The examples herein are provided to illustrate advantages of the present technology and to further assist a person of ordinary skill in the art with preparing or using the nanoparticles compositions of the present technology. To the extent that the compositions include ionizable components, salts such as pharmaceutically acceptable salts of such components may also be used. The examples herein are also presented in order to more fully illustrate the preferred aspects of the present technology. The examples should in no way be construed as limiting the scope of the present technology, as defined by the appended claims. The examples can include or incorporate any of the variations or aspects of the present technology described above. The variations or aspects described above may also further each include or incorporate the variations of any or all other variations or aspects of the present technology.

### EXAMPLES

### Materials and General Procedures

Materials. 1H-Imidazole-4-carboxylic acid, thionyl chloride (SOCl₂), tetraethyl orthosilicate (TEOS), tetrahydrofuran (THF), Triton X-100, acetone, ethanol, ammonia (30% in water) 1-ethyl-3-(3-dimethylaminopropyl)carbodiimide (EDC), and N-hydroxysuccinimide (NHS) were purchased from Fisher Scientific, USA. Hexanol, cyclohexane, 2-methyl-1H-imidazole (2-MIM), (3-aminopropyl)triethoxysilane (APTES), and DOX.HCl were bought from Tokyo Chemical Industry Co., Ltd., USA. Methoxypolyethylene glycol-N-succinimidyl ester (mPEG-NHS, Mₙ=5000) and hydroxyl-polyethylene glycol-N-succinimidyl ester (HO-PEG-NHS, Mₙ=5000) were obtained from Jenkem Technology, USA. Anhydrous zinc nitrate (ZnNO₃) was purchased from Sigma-Aldrich, USA. Nuclear localization signal (NLS)-tagged Streptococcus pyogenes Cas9 nuclease (sNLS-SpCas9-sNLS) was provided by Aldevron, USA.

Characterization. The chemical structures of TESPIC were analyzed by nuclear magnetic resonance (NMR) spectroscopy (Avance 400, Bruker Corporation, USA). The hydrodynamic diameter and zeta potential of the SMOF NPs were characterized by a dynamic light scattering (DLS) spectrometer (Malvern Zetasizer Nano ZS) at a 90° detection angle with a concentration at 0.1 mg/ml. The morphologies of SMOF NPs were characterized by transmission electron microscopy (TEM, Tecnai 12, Thermo Fisher, USA) and scanning electron microscope (SEM, Zeiss/LEO 1530, Carl Zeiss Microscopy, USA). X-ray powder diffraction of SMOF NPs were performed by Bruker D8 Discovery (Bruker Corporation, USA).

Cell Culture. Cells were cultured in a cell culture incubator (Thermo Fisher, USA) at 37° C. with 5% carbon dioxide at 100% humidity. HEK 293 cells (a human embryonic kidney cell line) including regular HEK 293 cells, GFP-expressing HEK cells, and BFP-expressing HEK cells, NHDF (a normal human dermal fibroblast cell line), and RAW 264.7 cells (a mouse macrophage cell line) were purchased from ATCC (USA) and cultured with DMEM medium (Gibco, USA) with 10% (v/v) fetal bovine serum (FBS, Gibco, USA) and 1% (v/v) penicillin-streptomycin (Gibco, USA). HCT 116 cells (a human colon cancer cell line) were cultured with 89% McCoy's 5 A medium, 10% FBS, and 1% penicillin-streptomycin.

Cell Viability Assay. HEK 293 cells were seeded onto 96 well plates (20,000 cells per well) 24 h prior to treatment. Cells were treated with complete medium, Lipo 2000 (0.5 μl/well), and empty SMOF NPs, whose concentrations ranged from 10 to 200 μg/ml. Cell viability was measured using a standard MTT assay 48 h after treatment (Thermo Fisher, USA).

Statistical Analysis. Results are presented as mean±standard deviation (SD). One-way analysis of variance (ANOVA) with Tukey's multiple comparisons was used to determine the difference between independent groups. Statistical analyses were conducted using GraphPad Prism software version 6.

### Example 1—Preparation of Silica Metal Organic Framework Nanoparticles (SMOF NPs)

Synthesis of N-(3-(triethoxysilyl)propyl)-1H-imidazole-4-carboxamide (TESPIC). 1H-Imidazole-4-carboxylic acid (500 mg, 3.85 mmol) solution in SOCl₂ (8 ml) was heated under stirring to reflux overnight before cooling to room temperature. The reaction mixture was cooled down to room temperature and added into toluene, and the precipitate was collected by filtration and dried in vacuo at room temperature to give the acid chloride intermediate, 1H-imidazole-4-carbonyl chloride. The freshly synthesized 1H-imidazole-4-carbonyl chloride was suspended in anhydrous THE (5 ml), and then triethylamine (855 mg, 8.47 mmol) and APTES (851 mg, 3.85 mmol) were added. The solution was stirred at room temperature overnight under a nitrogen atmosphere. The mixture was subsequently filtered, and the solvent was then removed by rotary evaporation to yield the final product TESPIC. Since the silica reactants have the tendency to undergo hydrolysis/polymerization during column purification, TESPIC was used without purification for SMOF NP formation.^{1,2 1}H NMR (400 MHz, DMSO-D6): δ 0.60 (dd, 2.4H, J=14.6, 6.2 Hz), δ 1.12 (t, 0H, J=7.0 Hz), δ 1.57 (dt, 2H, J=15.9, 8.0 Hz), δ 2.83-2.61 (m, 2H), δ 3.70 (q, 6H, J=6.0 Hz), δ 7.03 (s, 1H), δ 7.40 (s, 1H). ¹³C NMR (100 MHz, DMSO-D6): δ 165.76, 135.50, 132.89, 128.21, 58.01, 42.55, 22.88, 18.64, and 7.55.

Preparation of Silica-Metal-Organic-Framework Hybrid Nanoparticles (SMOF NPs). SMOF NPs were synthesized by a water-in-oil emulsion method. Triton X-100 (1.75 ml) and hexanol (1.75 ml) were dissolved in cyclohexane (7.5 ml) to form the organic phase. An aqueous ZnNO₃ (0.5 M) solution (20 μl) containing the desirable payload (e.g., DOX.HCl, DNA, mRNA, RNP, and RNP+ssODN; 5 mg/ml) was mixed with 400 μl of the organic phase. This mixture was vortexed for 15 s and then sonicated in an ultrasonic water bath for 15 s to form the water-in-oil emulsion, which was then magnetically stirred at 1500 rpm. To this emulsion, TEOS, TESPIC, APTES, and 2-MIM with different feed weight ratios were dissolved in 100 μl organic phase and added to the above emulsion. For example, to achieve a feed weight ratio of silica reactants (i.e., TEOS, TESPIC, APTES) to MOF reactant (i.e., 2-MIM) of 60:40, the total weight of TEOS+TESPIC+APTES added to the emulsion would be 1.2 mg, while the weight of 2-MIM would be 0.8 mg. Upon the addition of 3 μL of 30% ammonia aqueous solution, the mixture was stirred for 4 h at room temperature. Thereafter, mPEG-NHS (100 μg in 100 μl hexanol) was added to the above emulsion and was stirred for another 2 h. To prepare ATRA-modified SMOF NPs (i.e., SMOF-ATRA), HO-PEG-NHS was used instead of mPEG-NHS. The final payload-encapsulated SMOF NPs were precipitated by 600 μl acetone, and then washed by ethanol and water three times each.

For in vivo testing, the SMOF NPs were decorated with ATRA (i.e., SMOF-ATRA). ATRA binds to the inter-photoreceptor retinoid-binding protein, a major protein in the inter-photoreceptor matrix that selectively transports 11-cis-retinal to photoreceptor outer segments and all-trans-retinol to the RPE. ATRA was conjugated to the SMOF NP surface via EDC/NHS catalyzed esterification. Payload-encapsulated SMOF NPs (0.5 mg) were re-dispersed in 0.5 ml DI water. EDC (60 μg), NHS (60 μg) and a DMSO solution of ATRA (6 μg in 3 μl DMSO) were added to the above solution, and the pH was adjusted to 8. The solution was stirred at room temperature for 6 h followed by washing the SMOF-ATRA with water three times.

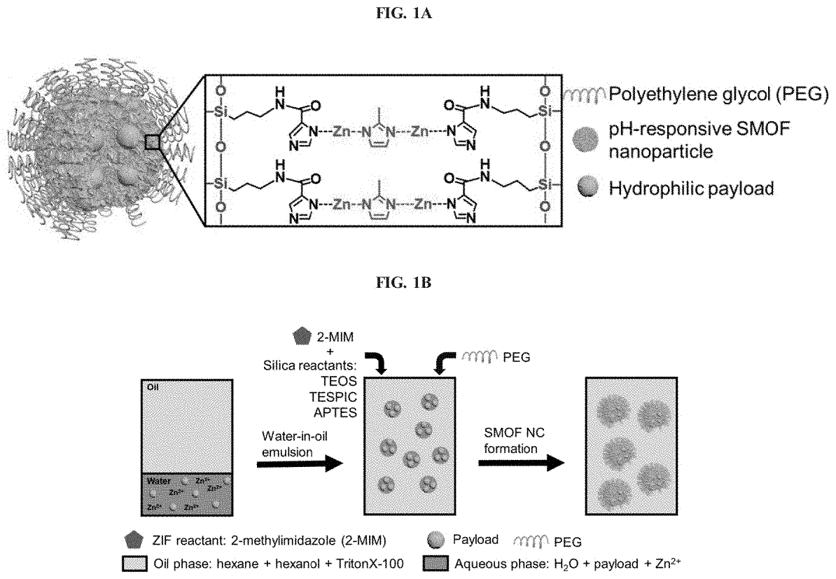

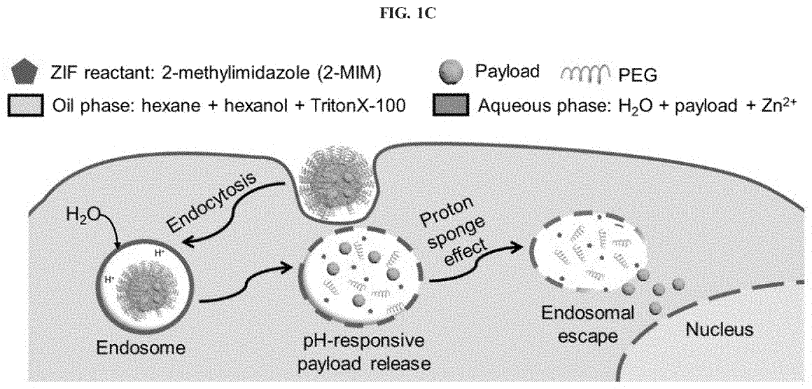

Results and Discussion. Silica-metal-organic-framework hybrid nanoparticles (SMOF NPs) were synthesized via a facile water-in-oil emulsion method (FIGS. 1A and 1B). An aqueous solution containing zinc ions at a constant concentration (0.5 M) and the desired payload was emulsified in the continuous oil phase, followed by additions of the silica reactants and the imidazole reactant (i.e., 2-methylimidazole (2-MIM)), which coordinates with zinc ions and forms the pH-responsive zeolitic imidazolate framework (ZIF)) (FIG. 1C). The silica reactive components included tetraethyl orthosilicate (TEOS), a basic building block that constructs the silica network, imidazole-containing N-(3-(triethoxysilyl)propyl)-1H-imidazole-2-carboxamide (TESPIC) that bridges the silica component with the ZIF component, and amine-containing (3-aminopropyl)triethoxysilane (APTES) that enables surface modification. Polyethylene glycol (PEG) was subsequently incorporated onto the SMOF NP surface after the formation of the SMOF NP, which allowed further surface functionalization (e.g., conjugation of targeting ligands and imaging agents). The as-prepared SMOF NPs were then collected by precipitation in acetone, centrifuged, and washed by ethanol and deionized (DI) water three times each to remove all residuals.

### Example 2—Physical Characterization of SMOF NPs

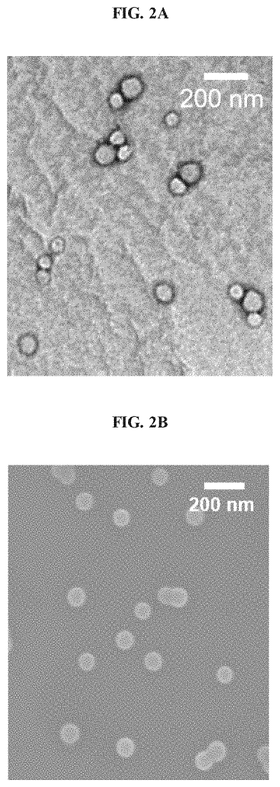

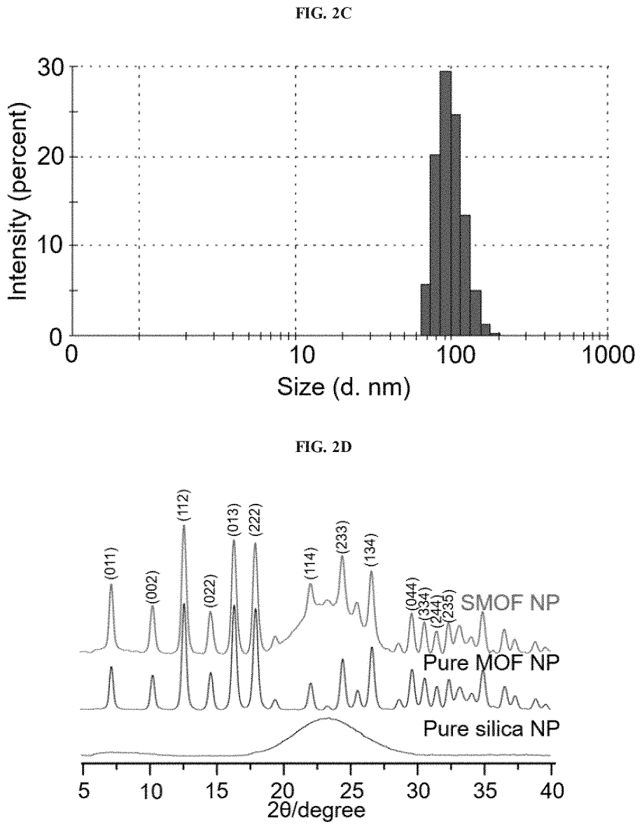

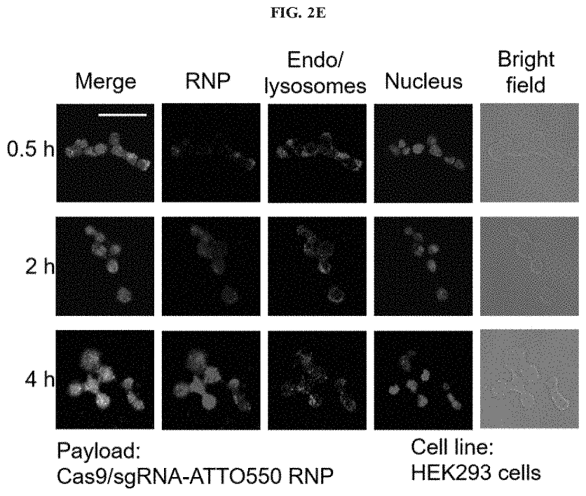

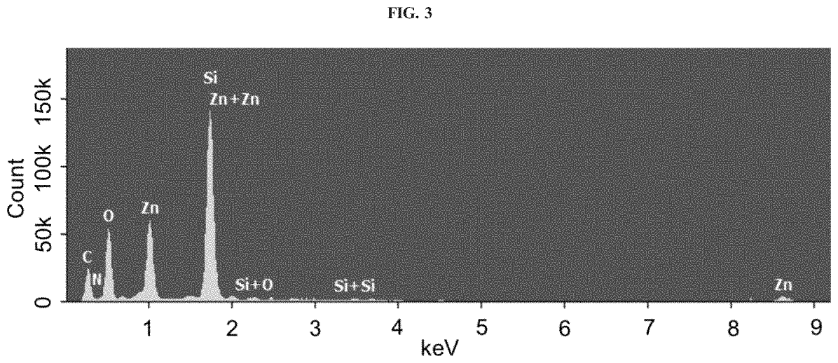

Morphology. The morphology of the DNA-loaded SMOF NP was characterized by scanning electron microscopy (SEM) and transmission electron microscopy (TEM). Spherical NPs with uniform sizes around 50-70 nm were observed, as shown in FIGS. 2A and 2B. The hydrodynamic diameter of DNA-loaded SMOF NPs, as measured by dynamic light scattering (DLS), was 110 nm (FIG. 2C). Zeta-potential measurements indicated that the DNA-loaded SMOF NPs had a slight positive surface charge (5.6±1 mV), similar to empty SMOF NPs (4.8 mV). Powder X-ray diffraction (XRD) spectra showed that SMOF NPs had similar crystal structures to ZIF (FIG. 2D). The ratio of the silica components and ZIF component in the SMOF NPs is controlled by the feed weight ratio of the silica reactants (i.e., TEOS, TESPIC, and APTES) and the ZIF reactant (i.e., 2-MIM). The ratio of the silica component and the ZIF component in the SMOF NPs was studied by energy-dispersive X-ray spectroscopy (EDS). As shown in FIG. 3, with the optimal feed weight ratio of the silica reactants to the ZIF reactant at 6:4, the elemental weight ratio between silicon (Si) and zinc (Zn) was 63:37 in the final SMOF NP, which is approximately equivalent to a 1:1 weight ratio of silica to ZIF.

Loading Content/Loading Efficiency Study. To calculate the loading content and loading efficiency of the payloads in SMOF NPs, 1 mg·mL⁻¹ of SMOF NP stock solution with different payloads were prepared. Thereafter, 10 μL of SMOF NP was incubated with 40 μL of acetate buffer (0.1 M, pH 5.5) for 30 min to allow the complete dissociation of SMOF NPs. The DOX.HCl loading content/efficiency was studied by UV-Vis spectroscopy. The RNP and RNP-ssODN loading contents/efficiencies were measured via a bicinchoninic acid assay (BCA assay, Thermo Fisher, USA). DNA and mRNA loading contents/efficiencies were evaluated using a NanoDrop One (Thermo Fisher, USA).

To investigate the versatility of SMOF NPs for the delivery of different hydrophilic payloads, including small molecule drugs (i.e., DOX.HCl), nucleic acids (i.e., DNA and mRNA), and CRISPR-Cas9 genome-editing machineries (i.e., RNP and RNP+ssODN (i.e., a combination of an RNP with a single-stranded oligonucleotide DNA (ssODN) donor template)), the loading content and loading efficiency of different payloads were quantified and summarized in Table 1. For small molecule DOX, the loading content was 17 wt %, with a loading efficiency of 92%. For hydrophilic biomacromolecules, the loading contents varied between 9.2-9.8 wt %, while the loading efficiencies ranged from 91-97%. The high loading contents and efficiencies can be contributed to the water-in-oil emulsion method that confined the payloads within the water droplet, followed by the formation of the SMOF NP network.

TABLE 1
Summary of loading content and loading efficiency
of different payloads by SMOF NPs.

| Payload | Loading content (wt %) | Loading efficiency (%) |
|---|---|---|
| DOX•HCl | 17 | 92 |
| DNA | 9.5 | 94 |
| mRNA | 9.2 | 91 |
| RNP | 9.8 | 97 |
| RNP + ssODN | 9.5 | 94 |

### Example 3—Hydrophilic Drug Delivery Studies

The cellular uptake behavior of DOX.HCl-loaded SMOF NPs was analyzed using flow cytometry. HEK 293 cells were seeded onto 96-well plates with 15,000 cells per well 24 h prior to treatment. The cells were then treated with free DOX.HCl and DOX.HCl-loaded SMOF NPs for 120 min with a DOX.HCl concentration of 5 μg/ml. Thereafter, cells were harvested with 0.25% trypsin-EDTA (Thermo Fisher, USA), spun down, and resuspended with 200 μl PBS (Thermo Fisher, USA). DOX.HCl uptake was obtained with an Attune NxT flow cytometer system (Thermo Fisher, USA) and analyzed with FlowJo 7.6.

The cytotoxicity of the DOX.HCl-loaded SMOF NPs was studied using an MTT assay. HEK 293 cells were seeded onto 96 well plates with 20,000 cells per well 24 h prior to treatment. The cells were then treated with free DOX.HCl, DOX.HCl-loaded SMOF NPs, and empty SMOF NPs (DOX.HCl concentrations of 6 and 12 μg/ml). Cells without treatment were used as a control group. After 48 h, the cell viability was measured using a standard MTT assay (Thermo Fisher, USA). Briefly, cells were treated with media containing 500 μg/ml MTT and incubated for 4 h. Then the MTT-containing media was aspirated. Next, the purple precipitates were dissolved in 150 μl of DMSO. The absorbance at 560 nm was obtained with a microplate reader (GloMax® Multi Detection System, Promega, USA).

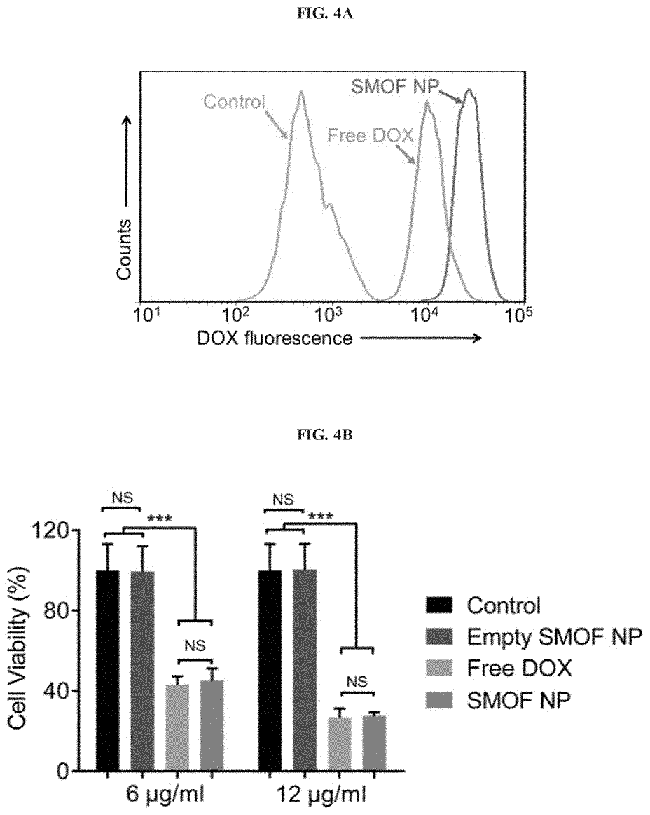

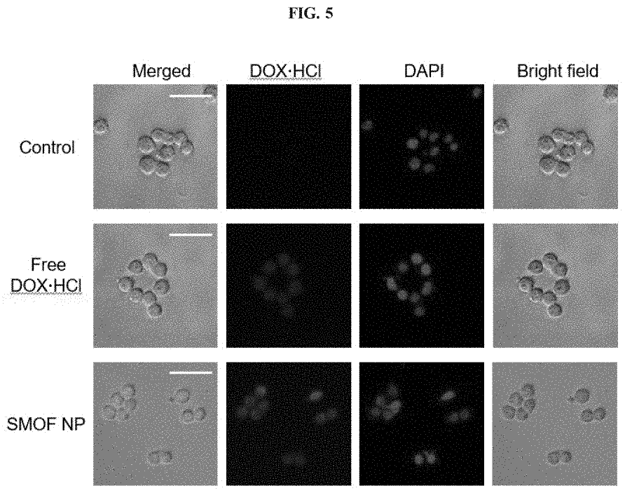

Efficient delivery of DOX.HCl via SMOF NPs was first studied by flow cytometry, in HEK293 cells, thus taking advantage of the fluorescence of DOX.HCl. Cells without DOX.HCl treatment were used as a control. As shown in FIG. 4A, DOX.HCl-loaded SMOF NPs exhibited a 3.2-fold higher level of DOX.HCl uptake than free DOX.HCl 2 h post treatment, indicating the efficient uptake of SMOF NPs by HEK293 cells. The cellular uptake of DOX.HCl-loaded SMOF NPs was also confirmed by fluorescence microscopy (FIG. 5). The therapeutic effect of DOX.HCl-loaded SMOF NPs was evaluated by an MTT assay (FIG. 4B). At both 6 μg/ml and 12 μg/ml DOX.HCl concentrations, the DOX.HCl-loaded SMOF NPs exhibited identical cytotoxicity to free DOX.HCl, while empty SMOF NPs showed no significant cytotoxicity. These results demonstrated the efficient delivery and release of DOX.HCl by SMOF NPs.

### Example 4—Transfection Studies

DNA Transfection Efficiency Study. HEK 293, HCT116, NHDF, and RAW 264.7 cells were seeded onto 96 well plates with 15,000 cells per well 24 h prior to treatment. Cells were transfected with green fluorescence protein (GFP) plasmid DNA (Addgene #40259, USA)-loaded SMOF NPs with a DNA dosage of 200 ng/well. DNA was also transfected using a commercially available transfection agent, Lipofectamine 2000 (i.e., Lipo 2000, Thermo Fisher, USA), as a positive control group. The amount of Lipo 2000 and DNA used per well was 0.5 μl and 200 ng, respectively. An untreated group was used as the negative control group. After 48 h, HEK 293, HCT116, and NHDF cells were harvested with 0.25% trypsin-EDTA, while RAW264.7 cells were harvested by repeated pipetting. The cells were then spun down and resuspended with 200 μl of PBS. GFP expression efficiencies were obtained with a flow cytometer and analyzed with FlowJo 7.6.

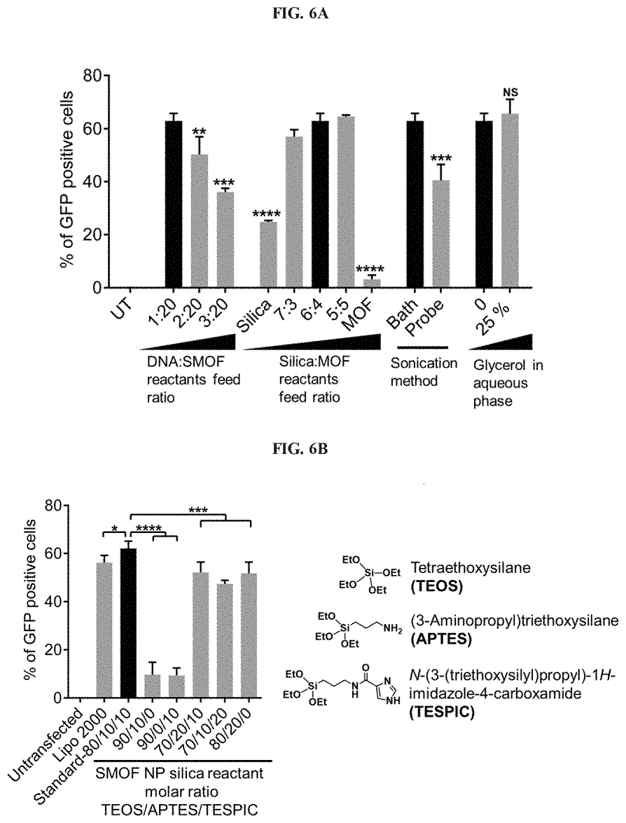

The SMOF NP formulation was first optimized in a human embryonic kidney (HEK 293) cell line using plasmid DNA as the payload (FIGS. 6A and 6B). Zinc ion concentration in the aqueous phase was fixed at 0.5 M, while various factors have been optimized including the feed ratio of the payload to the SMOF NP reactants, the feed ratio of the silica reactants (TEOS+APTES+TESPIC) to the MOF reactant (2-MIM), the emulsification process (e.g., bath sonication versus probe sonication), and the effect of an anti-freezing additive (i.e., glycerol) in the aqueous phase.

The feed weight ratio between the payload and the SMOF NP reactants is important, as insufficient SMOF NP forming materials may lead to a limited encapsulation volume and, subsequently, a low loading efficiency and the premature release and degradation of the payloads. On the other hand, too much MOF NP forming materials could result in insufficient/slow release of the payload within the target cells. For instance, SMOF NPs with a relatively lower feed ratio between DNA and the SMOF reactants (i.e., 1:20 by weight) exhibited a significantly higher transfection efficiency, thus indicating successful encapsulation of the payload within the SMOF NPs and an efficient intracellular release thereafter (FIG. 6A).

The feed weight ratio between the silica reactants (i.e., TEOS+APTES+TESPIC) and the MOF reactant (i.e., 2-MIM) is another critical factor for efficient payload delivery. Without the silica component, surface functionalization of the resulting MOF/ZIF NPs is very challenging as various functional groups can be conveniently introduced into the SMOF NPs through judicious selection of the silica reactants. Without the MOF component, silica NPs alone can neither escape endosomes and lysosomes efficiently nor release the payload rapidly in response to pH, thereby greatly minimizing the delivery efficiency. As shown in FIG. 6A, both pure silica NPs and pure MOF NPs formed via water-in-oil emulsions exhibited limited DNA transfection efficiencies. Moreover, pure MOF NPs showed larger particle sizes (data not shown) after purification, indicating inefficacious PEGylation and thus NP aggregation. The molar ratio of the three silica reactants—namely, TEOS, APTES, and TESPIC—was further optimized (FIG. 6B). The optimal formulation showing the highest DNA transfection efficiency was obtained when the molar ratio of TEOS:APTES:TESPIC was 80:10:10.

To evaluate the necessity of using a TEOS:APTES:TESPIC ternary composition to form the silica component in the SMOF NP instead of unary or binary counterparts, the SMOF NP formulation without TESPIC (i.e., TEOS:APTES:TESPIC, molar ratio of 90:10:0) was first tested. The resulting SMOF NPs exhibited significantly lower DNA transfection efficiencies, indicating TESPIC was essential for bridging the silica component to the MOF component within the hybrid SMOF NPs. Moreover, the formulation without APTES (i.e., TEOS:APTES:TESPIC, molar ratio of 90:0:10) also showed a limited transfection efficacy. However, higher APTES or TESPIC ratios didn't provide any advantage in achieving higher transfection efficiencies (FIG. 6B).

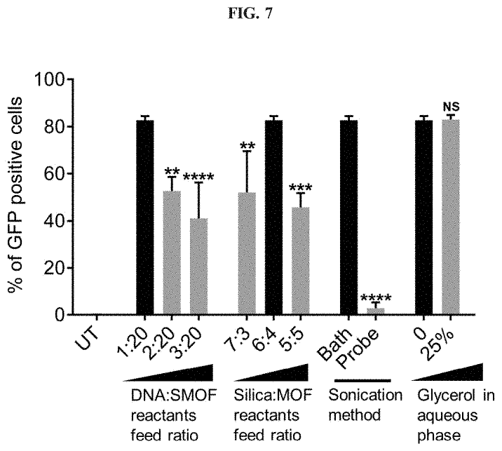

The sonication method also plays an important role in SMOF NP synthesis as it facilitates emulsification and controls the water droplet size in the emulsion. However, sonication that is too strong may affect the integrity of the biomacromolecular payload and thus reduce delivery efficiency. We found that using probe sonication for as short as 15 s can reduce the DNA transfection efficiency by 50% when compared with a gentler vortex+bath sonication method (FIG. 6A). Apart from sonication methods, additives in the aqueous phase, such as an anti-freezing agent (e.g., glycerol), may affect the SMOF NP formation and delivery efficiency. However, as shown in FIG. 6A, addition of glycerol up to 25% did not affect the formation of SMOF NP and or the transfection of DNA. These factors were further investigated in the mRNA-loaded SMOF NPs. While a similar trend was observed, the optimal feed ratio of the silica reactants to the MOF reactant is more critical in order to achieve efficient mRNA delivery in comparison with DNA Delivery (FIG. 7). Taken together, the optimal formulation of SMOF NPs was selected for further studies.

mRNA Transfection Efficiency Study. HEK 293, HCT116, NHDF, and RAW 264.7 cells were seeded onto 96 well plates with the density of 15,000 cells per well 24 h prior to treatment. Cells were transfected with GFP-mRNA (Ozbioscience, OZ Biosciences INC, San Diego, Calif.)-loaded SMOF NPs, with an mRNA dosage of 200 ng/well. mRNA was also transfected using a commercially available transfection agent, Lipofectamine 2000 (i.e., Lipo 2000), as a positive control group. The amount of Lipo 2000 and mRNA used per well was 0.5 μl and 200 ng, respectively. An untreated group was used as the negative control group. After 48 h, HEK 293, HCT116, and NHDF cells were harvested with 0.25% trypsin-EDTA, while RAW264.7 cells were harvested by repeated pipetting. The cells were spun down and resuspended with 200 μl of PBS. GFP expression efficiencies were obtained with a flow cytometer and analyzed with FlowJo 7.6.

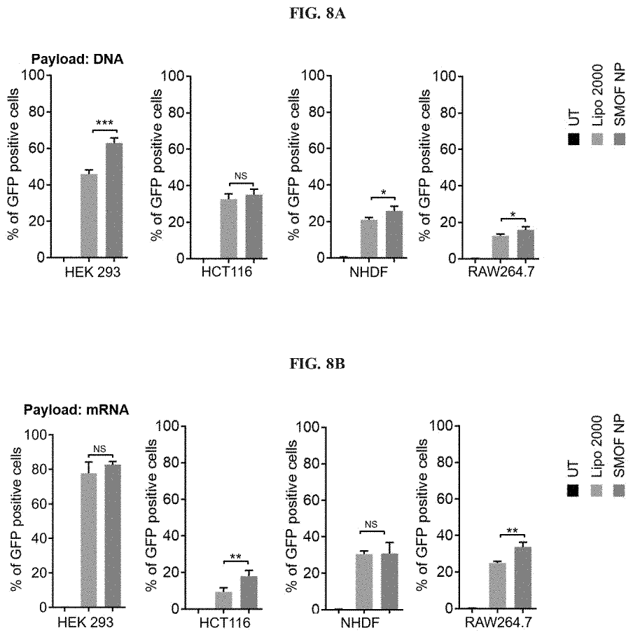

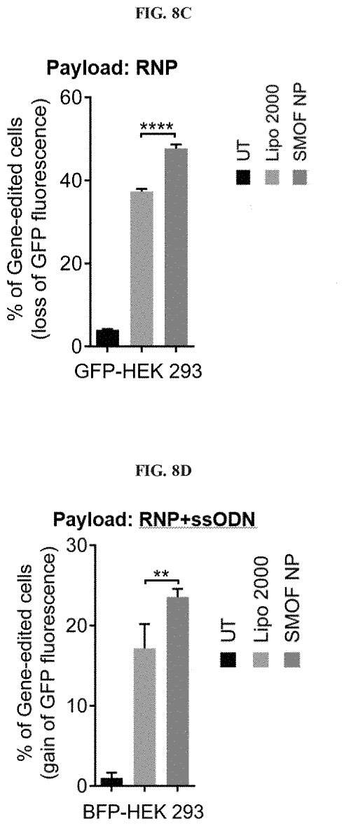

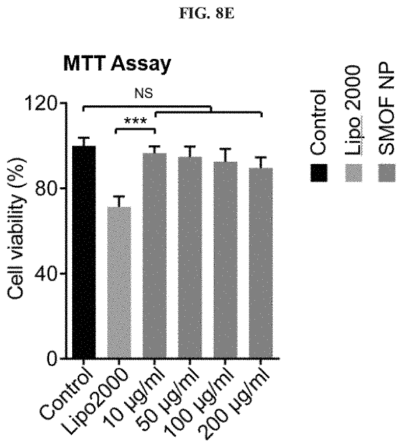

The transfection efficiency of DNA-loaded SMOF NPs was studied in 4 different cell types, including a HEK293 cell line, a human colon tumor (HCT116) cell line, a human normal dermal fibroblast (NHDF) cell line, and a rat macrophage (RAW264.7) cell line. In HCT116 cells, DNA-loaded SMOF NPs exhibited a similar transfection efficiency to the commercially available transfection agent Lipofectamine 2000 (Lipo2000), while in the other 3 cell lines, SMOF NP showed significantly higher transfection efficiencies than Lipo2000 (FIG. 8A). For mRNA-loaded SMOF NPs, they showed similar transfection efficiencies to Lipo2000 in HEK293 and NHDF cells, but significantly higher transfection efficiencies in HCT116 and RAW264.7 cells (FIG. 8B), indicating the efficient delivery of nucleic acids by SMOF NPs.

### Example 5—Gene Editing Studies

RNP Genome-Editing Efficiency Study. GFP-expressing HEK 293 cells were used as an RNP transfection cell model. Cells were seeded onto 96 well plates at 5,000 cells per well 24 h prior to treatment. RNP was prepared as previously reported⁶ by mixing sNLS-SpCas9-sNLS and in vitro transcribed sgRNA (GFP protospacer: GCACGGGCAGCTTGCCGG (SEQ ID NO: 41)) at a 1:1 molar ratio. Cells were treated with Lipo 2000 (0.5 μl/well) and then complexed with RNP or RNP-loaded SMOF NPs. An untreated group was used as the control group. A quantity of 100 μl of fresh culture medium was added into each well 48 h after treatment and thereafter, half of the culture medium was refreshed every 48 h. Six days after treatment, cells were harvested with 0.25% trypsin-EDTA, spun down, and re-suspended with 200 μl of PBS. The RNP genome-editing efficiencies were quantified via flow cytometry. Data were analyzed with FlowJo 7.6.

RNP+ssODN Co-Delivery for Precise Gene Correction. The RNP+ssODN mixture was prepared by simply mixing the as-prepared RNP and single-stranded oligonucleotide DNA (ssODN) donor template at 4° C. for 5 min at a 1:1 molar ratio. Blue fluorescence protein (BFP)-expressing HEK 293 cells generated through lentiviral transduction of a BFP dest clone (Addgene, Cambridge, Mass.) were employed as a model cell line³. When cells are transfected with RNP+ssODN-targeting BFP (BFP protospacer: GCTGAAGCACTGCACGCCAT (SEQ ID NO: 42)), if precise editing occurs, three nucleotides within the BFP gene are edited and converted to a green fluorescent protein (GFP) gene as described previously.⁶ BFP-expressing HEK 293 cells were seeded onto a 96 well plate at 15,000 cells per well 24 h prior to treatment. Cells were treated with Lipo 2000 (0.5 μl/well) loaded with RNP and ssODN or with RNP+ssODN-loaded SMOF NPs. For each treatment, the RNP+ssODN dosage was kept at 150 ng/well (i.e., an equivalent Cas9 protein dosage of 125 ng/well). The precise gene-editing efficiencies were quantified 6 days after treatment using flow cytometry by counting the percentage of green fluorescence positive cells. Data were analyzed with FlowJo 7.6.

Cas9 can cleave double-stranded DNA from a specific genomic locus under the guidance of sgRNA. After the double-stranded DNA break is generated, gene deletion can be achieved by the nonhomologous end-joining (NHEJ) DNA repair pathway.⁴ To investigate the genome editing efficiency of RNP-loaded SMOF NPs, an sgRNA targeting the mCherry gene in a transgenic GFP-expressing HEK 293 cell line was used. To enhance the nuclear transportation, a Cas9 protein fused with two NLS peptides (sNLS-Cas9-sNLS) was used to form the RNP complexes. As shown in FIG. 8C, SMOF NPs exhibited a similar GFP gene knockout efficiency to Lipo2000. Furthermore, to achieve precise genome editing by co-delivery of RNP and a donor DNA template, gene correction or insertion can be achieved through the homology-directed repair (HDR) pathway.⁵ RNP and a donor single-stranded oligonucleotide DNA (ssODN)(BFP to GFP ssODN sequence: 5′-TCATGTGGTCGGGGTAGCGGCTGAAGCACTGCACGCCATGG GTCAGGGTGGTCACGAGGGTGGGCCAGGGCACCGGCAGCTTGCCGGTGGTGCAGAT GAA-3′ (SEQ ID NO: 43), changing BFP to GFP via alternation of histidine to tyrosine) were loaded into SMOF NPs and the precise genome-editing efficiency was studied using BFP-expressing HEK293 cells. Precise gene editing will lead to the replacement of 3 nucleotides in the genome, thereby converting BFP to GFP. The precise genome-editing efficiency was evaluated by the percentage of GFP positive cells. As shown in FIG. 8D, SMOF NPs showed a significantly higher gene correction efficiency than Lipo2000. These studies indicate that SMOF NPs are suitable for the delivery of CRISPR genome-editing machineries.

Compatibility Studies. To study the biocompatibility of SMOF NPs, the cells were treated with SMOF NPs and the cell viability was investigated by an MTT assay. SMOF NPs did not induce significant cytotoxicity in HEK cells with concentrations up to 200 μg/ml, which was at least 9-fold of the concentration used for our studies (FIG. 8E). Similar to previous reports, Lipo2000 exhibited significant cytotoxicity (with 30% cell death) at the dosage indicated in the user's manual (i.e., 5 μg/ml).⁷

Intracellular Trafficking of RNP SMOF NPs. Intracellular trafficking of RNP SMOF NPs was investigated by confocal laser scanning microscopy (CLSM, Nikon, Japan). In this case, ATTO550-labeled sgRNA was used to form the Cas9/ATTO-sgRNA RNP loaded into the SMOF NPs. HEK 293 cells were seeded onto a Nunc™ Lab-Tek™ II CC2™ Chamber Slide (Thermo Fisher, USA) at 50,000 cells per well 24 h prior to treatment. At each time point (i.e., 0.5, 2, and 4 h) after SMOF NP treatments, the cells were washed by PBS, and then stained with endosome/lysosome marker LysoTracker Green DND-26 (100 nM) and nucleus marker Hoechst 33342 (10 μg/mL) for 30 min at 37° C.

The SMOF NP contains a pH-responsive ZIF component that degrades in acidic environments, leading to a rapid release of the payload.⁸ Meanwhile, the ZIF component can also facilitate the endosomal escape of the payload because the imidazole groups (pKa˜6.0) can be protonated in the acidic endocytic compartments (i.e., endosomes), leading to endosomal-membrane disruption by the proton sponge effect. To study the intracellular trafficking of SMOF NPs, confocal laser scanning microscopy (CLSM) was used to image the subcellular distribution of Cas9/ATTO550-labeled sgRNA ribonucleoprotein (RNP) delivered by SMOF NPs (FIG. 2E). RNP was observed to co-localize with endo/lysosomes 0.5 h post-treatment, indicating that the uptake of RNP-loaded SMOF NPs occurred via endocytosis. The extent of co-localization of RNP and endo/lysosomes decreased 2 h post-treatment, indicating the efficient endo/lysosomal escape capabilities of SMOF NPs. Assisted by a nuclear localization signal (NLS) fused on both terminuses of the Cas9 nuclease, the RNP signal showed considerable overlap with the nucleus and minimal co-localization with endo/lysosomes as early as 4 h post-treatment, thus indicating the efficient escape from endo/lysosomes and the successful nuclear transportation of RNP.

### Example 6—In Vivo Eye Study

All animal research was approved by UW-Madison animal care and use committee. For in vivo studies, Ai14 reporter mice (obtained from The Jackson Laboratory) were used to assess the genome editing efficiency induced by RNP-loaded SMOF NPs. RNPs were prepared using either a sgRNA targeting the stop cassette composed of 3 SV40 polyA blocks (target sequence: 5′-AAGTAAAACCTCTACAAATG-3′ (SEQ ID NO: 44)) in Ai14 mice or a mouse negative control sgRNA (Integrated DNA Technologies, guide sequence: CGTTAATCGCGTATAATACG (SEQ ID NO: 45)). Subretinal injection and subsequent RPE tissue collection were performed as reported.⁹ Mice were maintained under tightly controlled temperature (23±5° C.), humidity (40-50%), and light/dark (12/12 h) cycle conditions under a 200 lux light environment. The mice were anesthetized by intraperitoneal injection of a ketamine (80 mg/kg), xylazine (16 mg/kg) and acepromazine (5 mg/kg) cocktail. Before the subretinal injection, the cornea was anesthetized with a drop of 0.5% proparacaine HCl, and the pupil was dilated with 1.0% tropicamide ophthalmic solution. Mice were placed on a temperature-regulated heating pad during the injection and for recovery purposes. All surgical manipulations were carried out under a surgical microscope (AmScope, Irvine, Calif.). Right eyes of mice were injected with SMOF-ATRA encapsulating RNP with a sgRNA targeting the Ai14 stop cassette, left eyes of mice were injected with SMOF-ATRA encapsulating RNP with a negative control sgRNA. Two microliters of SMOF-ATRA solutions containing 4 μg RNP was injected into the subretinal space using a UMP3 ultramicro pump fitted with a NanoFil syringe, and the RPE-KIT (all from World Precision Instruments, Sarasota, Fla.) equipped with a 34-gauge beveled needle. Successful administration was confirmed by visualization of bleb formation. The tip of the needle remained in the bleb for 10 s after bleb formation, and then gently withdrawn.

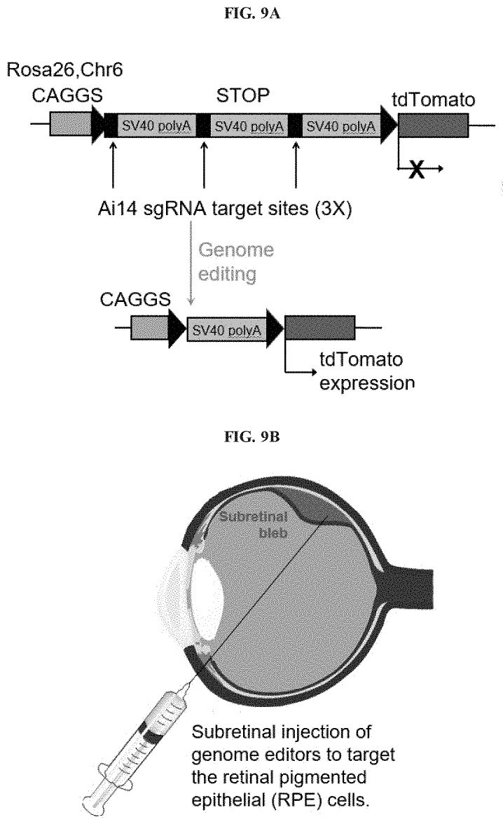

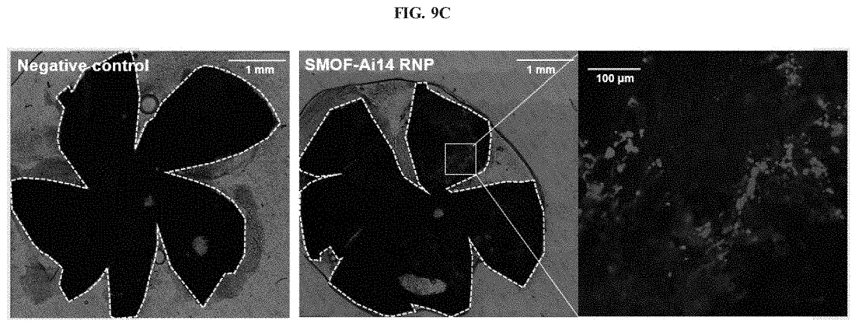

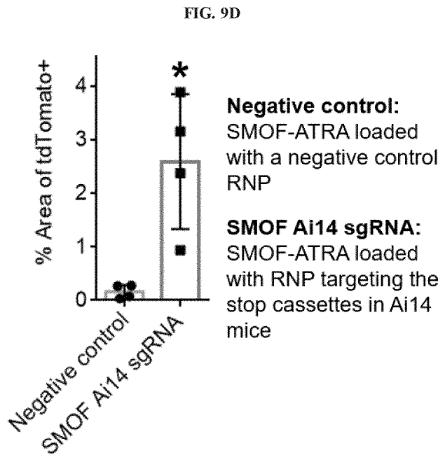

All cells of an Ai14 mouse contain a CAGGS promoter and a loxP-flanked stop cassette (three repeats of the SV40 polyA sequence) that prevents expression of the tdTomato fluorescent protein at the Rosa26 locus. The gain-of-function fluorescent signal in modified cells provided a robust and quantitative readout of genome editing at the stop cassette.⁹⁻¹¹ As shown in FIG. 9A, RNP targeting the excision of the SV40 polyA blocks can induce tdTomato expression. The genome editing efficiency of the RNP-loaded SMOF NPs could be easily monitored through fluorescence imaging. The genome editing efficiency was studied within the targeted retinal pigmented epithelium (RPE) of the Ai14 mice (as discussed above RPE abnormality can cause a variety of eye diseases, e.g., retinal degeneration and blindness).¹² SMOF NPs were decorated with ATRA (i.e., SMOF-ATRA), which binds to the inter-photoreceptor retinoid-binding protein—a major protein in the inter-photoreceptor matrix that selectively transports 11-cis-retinal to photoreceptor outer segments and all-trans-retinol to the RPE.¹³⁻¹⁴ Mice were subretinally injected with SMOF-ATRA NPs loaded with RNPs targeting the Ai14 stop cassette (right eye) and SMOF-ATRA NPs loaded with negative control RNPs (left eye) (FIG. 9B). To assess tdTomato expression generated by successful genome editing, the mice were sacrificed, and eyes were collected 13 days after injection and rinsed twice with PBS. A puncture was made at ora serrata with an 18-gauge needle, and the eye was opened along the corneal incisions. The lens was then removed. The entire RPE tissue was separated from the enucleated eye by radially incising the eyecup to the center and flattening to give a final floret shape. The RPE layer was then separated and flat-mounted on a cover-glass slide to assess genome editing via CLSM. RPE tissues were imaged with a NIS-Elements using a Nikon C2 confocal microscope (Nikon Instruments Inc.). ImageJ (NIH) was used for image analysis.

As shown in FIGS. 9C and 9D, strong tdTomato signals were visualized in the eyes injected with the RNP-loaded SMOF-ATRA targeting the Ai14 stop cassette, while little tdTomato signal was found in eyes treated with negative control SMOF-ATRA (i.e., SMOF-ATRA encapsulating RNP with negative control sgRNA). These results indicate successful delivery of RNP and robust in vivo genome editing induced by SMOF-ATRA.

### REFERENCES

1. Yuan, P., Zhang, H., Qian, L., Mao, X., Du, S., Yu, C., Peng, B., and Yao, S. Q. Intracellular Delivery of Functional Native Antibodies under Hypoxic Conditions by Using a Biodegradable Silica Nanoquencher. Angew. Chem. 2017, 129, 12655-12659.
2. Yuan, P., Mao, X., Chong, K. C., Fu, J., Pan, S., Wu, S., Yu, C., and Yao, S. Q. Simultaneous Imaging of Endogenous Survivin Mrna and on-Demand Drug Release in Live Cells by Using a Mesoporous Silica Nanoquencher. Small 2017, 13, 1700569.
3. S. K. Alsaiari, S. Patil, M. Alyami, K. Alamoudi, f. Aleisa, J. Merzaban, M. Li, N. M. Khashab, J. Am. Chem. Soc. 2018, 140, 1, 143-146.
4. a) J. A. Doudna, E. Charpentier, Science 2014, 346, 1258096; b) L. Cong, F. A. Ran, D. Cox, S. Lin, R. Barretto, N. Habib, P. D. Hsu, X. Wu, W. Jiang, L. Marraffini, Science 2013, 1231143.
5. J. Carlson-Stevermer, A. A. Abdeen, L. Kohlenberg, M. Goedland, K. Molugu, M. Lou, K. Saha, Nat. Commun. 2017, 8, 1711.
6. Carlson-Stevermer, J., Abdeen, A. A., Kohlenberg, L., Goedland, M., Molugu, K., Lou, M., and Saha, K. Assembly of Crispr Ribonucleoproteins with Biotinylated Oligonucleotides Via an Rna Aptamer for Precise Gene Editing. Nat. Commun. 2017, 8, 1711.
7. Y. Wang, B. Ma, A. A. Abdeen, G. Chen, R. Xie, K. Saha, S. Gong, ACS Appl. Mater. Interfaces 2018, 10, 31915.
8. S. K. Alsaiari, S. Patil, M. Alyami, K. Alamoudi, Fi. Aleisa, J. Merzaban, M. Li, N. M. Khashab, Endosomal Escape and Delivery of CRISPR/Ca9 Genome Editing Machinery Enabled by Nanoscale Zeolithic Imidazolate Framework. J. Am. Chem. Soc. 2018, 140, 1, 143-146.
9. Chen, G.; Abdeen, A. A.; Wang, Y.; Shahi, P. K.; Robertson, S.; Xie, R.; Suzuki, M.; Pattnaik, B. R.; Saha, K.; Gong, S., A Biodegradable Nanocapsule Delivers a Cas9 Ribonucleoprotein Complex for in Vivo Genome Editing. Nature Nanotechnology 2019.
10. Staahl, B. T.; Benekareddy, M.; Coulon-Bainier, C.; Banfal, A. A.; Floor, S. N.; Sabo, J. K.; Urnes, C.; Munares, G. A.; Ghosh, A.; Doudna, J. A., Efficient Genome Editing in the Mouse Brain by Local Delivery of Engineered Cas9 Ribonucleoprotein Complexes. Nat. Biotechnol. 2017, 35 (5), 431.
11. Madisen, L.; Zwingman, T. A.; Sunkin, S. M.; Oh, S. W.; Zariwala, H. A.; Gu, H.; Ng, L. L.; Palmiter, R. D.; Hawrylycz, M. J.; Jones, A. R., A Robust and High-Throughput Cre Reporting and Characterization System for the Whole Mouse Brain. Nat. Neurosci. 2010, 13 (1), 133.
12. Berger, W.; Kloeckener-Gruissem, B.; Neidhardt, J., The Molecular Basis of Human Retinal and Vitreoretinal Diseases. Prog Retin Eye Res 2010, 29 (5), 335-375.
13. Sun, D.; Sahu, B.; Gao, S.; Schur, R. M.; Vaidya, A. M.; Maeda, A.; Palczewski, K.; Lu, Z.-R., Targeted Multifunctional Lipid Eco Plasmid DNA Nanoparticles as Efficient Non-Viral Gene Therapy for Leber's Congenital Amaurosis. Molecular Therapy—Nucleic Acids 2017, 7, 42-52.
14. Carlson, A.; Bok, D., Promotion of the Release of 11-Cis-Retinal from Cultured Retinal Pigment Epithelium by Interphotoreceptor Retinoid-Binding Protein. Biochemistry—us 1992, 31 (37), 9056-9062.

### EQUIVALENTS

While certain embodiments have been illustrated and described, a person with ordinary skill in the art, after reading the foregoing specification, can effect changes, substitutions of equivalents and other types of alterations to the nanoparticles of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1. A nanoparticle comprising:
a organosilica network comprising a plurality of imidazolyl groups and/or carboxyl groups, wherein the organosilica network further comprises a plurality of surface-modifying moieties selected from the group consisting of polyethylene glycol (PEG), a polycation, a polyzwitterion, or functional groups that form cations at a pH of 8 or below;
a metal organic framework component comprising a transition metal ion coordinated to a coordinating ligand, wherein the transition metal ion is selected from the group consisting of zinc, iron, zirconium, copper, and cobalt ions, and the coordinating ligand is selected from an imidazolate ligand or a carboxylate ligand;
a bioactive payload selected from the group consisting of a hydrophilic drug, a polynucleic acid, a protein and a protein-polynucleic acid complex;
wherein the nanoparticle comprises an exterior surface with a plurality of surface-modifying groups.

2. The nanoparticle of claim 1, wherein the organosilica network comprises imidazolyl groups.

3. The nanoparticle of claim 1, wherein the metal is zinc.

4. The nanoparticle of claim 1, wherein the coordinating ligand is selected from the group consisting of imidazole, 2-methyl-imidazole, benzimidazole, 5-methylbenzimidazole, terephthalic acid, 2-methyl-pterphthalic acid, 2-hydroxy-terephthalic acid, and 2-amino-terephthalic acid, benzene-1,3,5-tricarboxylic acid, 1,3,5-tris(4-carboxyphenyl)benzene, 2,6-naphthalenedicarboxylic acid, 4,4′,4″-s-triazine-2,4,6-triyl-tribenzoic acid, 2,5-dihydroxyterephthalic acid.

5. The nanoparticle of claim 1, wherein the surface-modifying moieties comprise functional groups that form cations at a pH of 8 or below and are selected from the group consisting of amino, guanidine, and pyridyl.

6. The nanoparticle of claim 1, wherein the surface-modifying moieties comprise polycations selected from the group consisting of polyethyleneimine (PEI), polylysine, and polyamidoamine (PAMAM), and having a Mn of about 1,000 to about 50,000 Da.

7. The nanoparticle of claim 1, wherein the surface-modifying moieties comprise polyzwitterions selected from the group consisting of poly(carboxybetaine methacrylate), poly(sulfobetain methacrylate), and poly(2-methacryloyloxyethyl phosphorylcholine), and having a Mn of about 1,000 to about 50,000 Da.

8. The nanoparticle of claim 1, wherein the surface-modifying moieties comprise polyethylene glycol (PEG) having a Mn of about 1,000 to about 50,000 Da.

9. The nanoparticle of claim 8 wherein the PEG has an Mn of about 2,000 to about 10,000 Da.

10. The nanoparticle of claim 1 wherein the weight ratio of organosilica network to metal organic framework component ranges from 3:1 to 1:3.

11. The nanoparticle of claim 1 further comprising a targeting ligand and/or an imaging agent attached to the organosilica network.

12. The nanoparticle of claim 11, wherein the targeting ligand and/or imaging agent are attached to the organosilica network via bonds to organosilica network amino groups.

13. The nanoparticle of claim 1, wherein the payload is selected from doxorubicin or a salt thereof, DNA, RNA, ribonucleoprotein (RNP), and combinations of two or more thereof.

14. The nanoparticle of claim 1, wherein the payload is selected from doxorubicin or the salt thereof, ribonucleoprotein (RNP), plasmid DNA (pDNA), single-stranded donor oligonucleotide (ssODN), complementary (cDNA), messenger RNA (mRNA), small interfering RNA (siRNA), microRNA (miRNA), short hairpin RNA (shRNA), single guide RNA (sgRNA), transfer RNA (tRNA), ribozymes, and combinations of two or more thereof.

15. The nanoparticle of claim 1, wherein the payload is Cas9 RNP or RNP+ssODN.

16. The nanoparticle of claim 1, having an average hydrodynamic diameter of 10 to 500 nm.

17. A nanoparticle comprising:
a organosilica network comprising a plurality of imidazolyl groups, wherein the organosilica network further comprises a plurality of amino groups as surface-modifying groups;
a metal organic framework component comprising zinc ion and 2-methylimidazolate;
a bioactive payload selected from the group consisting of a hydrophilic drug, a polynucleic acid, a protein, and a protein-polynucleic acid complex; and
PEG conjugated to at least some of the organosilica network amino groups, and forming at least part of the exterior surface of the nanoparticle; and
wherein the zinc coordinates to one or both of the 2-methylimidazolate and the organosilica network imidazolyl groups.

18. A method of making a nanoparticle of any one of the preceding claims comprising:
forming a nanoparticle comprising a organosilica network by adding organosilica network precursors and an organic framework component to an emulsion of water and an organic solvent, whereby the organosilica network precursors polymerize to form the organosilica network, and wherein
the organosilica network precursors include imidazolyl groups and/or carboxyl groups and/or functional groups that form cations at a pH of 8 or below, and
the emulsion comprises a metal ion and a bioactive payload, wherein the metal is selected from the group consisting of zinc, iron, zirconium, copper, and cobalt, and the bioactive payload selected from the group consisting of a hydrophilic drug, a polynucleic acid, a protein and a protein-polynucleic acid complex.

19. The method of claim 18 wherein the organosilica network precursors comprise tetraethyl orthosilicate, N-(3-(triethoxysilyl)propyl)-1H-imidazole-2-carboxamide and (3-aminopropyl)triethoxysilane.

20. The method of claim 18 further comprising attaching a surface-modifying moiety to the organosilica network, wherein the surface-modifying moiety is selected from the group consisting of PEG, a polycation, and a polyzwitterion.

21. A method of delivering a bioactive payload to a targeted cell comprising exposing the targeted cell to the nanoparticle of claim 1.

22. The method of claim 21 comprising administering the nanoparticle or a composition comprising the nanoparticle to a subject in need thereof.

23. The method of claim 22 wherein the subject is a human.

24. The method of claim 21, wherein the payload is doxorubicin or the salt thereof, DNA, mRNA, Cas9 RNP, or RNP+ssODN.

25-27. (canceled)
